(19) United States
(12) Patent Application Publication
(10) Pub. No.: US 2023/0272390 A1
(43) Pub. Date: Aug. 31, 2023

(54) COMPOSITIONS AND METHODS FOR HEMOGLOBIN PRODUCTION

(71) Applicants: THE CHILDREN'S HOSPITAL OF PHILADELPHIA, Philadelphia, PA (US); THE TRUSTEES OF THE UNIVERSITY OF PENNSYLVANIA, Philadelphia, PA (US)

(72) Inventors: Gerd Blobel, Bala Cynwyd, PA (US); Xianjiang Lan, Aldan, PA (US); Junwei Shi, Philadelphia, PA (US)

(21) Appl. No.: 18/015,800

(22) PCT Filed: Jul. 29, 2021
(86) PCT No.: PCT/US21/43606

§ 371 Date: Jan. 12, 2023

Related U.S. Application Data
(60) Provisional application No. 63/058,065, filed on Jul. 29, 2020.

(51) Int. Cl. C12N 15/113; A61K 31/454

(52) U.S. Cl. CPC C12N 15/113; A61K 31/454; C12N 2310/16; C12N 2310/14; C12N 2310/531; C12N 2310/11

(57) ABSTRACT

Methods and compositions for producing fetal hemoglobin and treating a hemoglobinopathy or thalassemia are disclosed. Specifically, the disclosure provides methods of increasing the level of human fetal hemoglobin and/or gamma-globin in a cell or subject, the method comprising administering at least one zinc finger protein 410 (ZNF410) inhibitor to the cell or subject, wherein the subject has a beta-chain hemoglobinopathy, thalassemia, and/or sickle cell disease.

This application is a § 371 application of PCT/US2021/043606, filed Jul. 29, 2021, which claims priority under 35 U.S.C. § 119(e) to U.S. Provisional Patent Application No. 63/058,065, filed Jul. 29, 2020. The foregoing applications are incorporated by reference herein.

This invention was made with government support under Grant Nos. R01HL119479 and DK106766 awarded by National Institutes of Health. The government has certain rights in the invention.

Incorporated herein by reference in its entirety is the Sequence Listing being concurrently submitted via EFS-Web as a text file named SeqList.txt, created Jan. 5, 2023, and having a size of 27,664 bytes.

### FIELD OF THE INVENTION

The present invention relates to the field of hematology. More specifically, the invention provides compositions and methods for the production of various forms of hemoglobin, including adult and fetal type hemoglobin.

### BACKGROUND OF THE INVENTION

Several publications and patent documents are cited throughout the specification in order to describe the state of the art to which this invention pertains. Each of these citations is incorporated herein by reference as though set forth in full.

Sickle cell disease and thalassemia cause significant worldwide morbidity and mortality (Modell et al. (2008) Bull. World Health Org., 86:480-487; Modell et al. (2008) J. Cardiovasc. Magn. Reson., 10:42). However, effective drugs do not exist for these illnesses. One goal in the treatment of these diseases is to reactivate fetal hemoglobin (HbF). HbF reduces the propensity of sickle cell disease red blood cells to undergo sickling. Indeed, high fetal globin levels are associated with improved outcomes for sickle cell anemia patients (Platt et al. (1994) N. Engl. J. Med., 330: 1639-1644). Elevating HbF also reduces the globin chain imbalance in certain thalassemias, thereby improving symptoms. There is an enormous unmet need to identify compounds that ameliorate the course of these diseases.

### SUMMARY OF THE INVENTION

In accordance with the present invention, compositions and methods are provided for increasing hemoglobin levels (e.g., fetal hemoglobin) and/or γ-globin in a cell or subject. In a particular embodiment, the method comprises administering at least one zinc finger protein 410 (ZNF410) inhibitor to the cell or subject. In a particular embodiment, the subject has a hemoglobinopathy or thalassemia. In a particular embodiment, the cell is an erythroid cell. In a particular embodiment, the ZNF410 inhibitor is a small molecule. The ZNF410 inhibitor may be, for example, a DNA binding domain inhibitor or a polypeptide comprising at least four zinc fingers of ZNF410. The ZNF410 inhibitor may be an inhibitory nucleic acid molecule. In a particular embodiment, the ZNF410 inhibitor is CRISPR based and targets the ZNF410 gene. In a particular embodiment, the ZNF410 inhibitor is an siRNA or shRNA targeting a nucleic acid encoding ZNF410. In a particular embodiment, the ZNF410 inhibitor is a proteolysis-targeting chimera (PROTAC) based small molecule targeting the ZNF410 protein for degradation. The method may further comprise delivering at least one fetal hemoglobin inducer to the cell or subject. The method may exploit additive or synergistic effects with other fetal hemoglobin inducing methods based on pharmacologic compounds or various forms of gene therapy.

In accordance with another aspect of the instant invention, methods of inhibiting, treating, and/or preventing a hemoglobinopathy (e.g., sickle cell disease) or thalassemia in a subject are provided. In a particular embodiment, the method comprises administering at least one ZNF410 inhibitor to a subject in need thereof. The ZNF410 inhibitor may be in a composition with a pharmaceutically acceptable carrier. In a particular embodiment, the subject has a β-chain hemoglobinopathy. In a particular embodiment, the subject has sickle cell anemia. In a particular embodiment, the ZNF410 inhibitor is a small molecule. The ZNF410 inhibitor may be, for example, a DNA binding domain inhibitor or a polypeptide comprising at least four zinc fingers of ZNF410. The ZNF410 inhibitor may be an inhibitory nucleic acid molecule. In a particular embodiment, the ZNF410 inhibitor is CRISPR based and targets the ZNF410 gene. In a particular embodiment, the ZNF410 inhibitor is an siRNA or shRNA targeting a nucleic acid encoding ZNF410. In a particular embodiment, the ZNF410 inhibitor is a proteolysis-targeting chimera (PROTAC) based small molecule targeting the ZNF410 protein for degradation. The method may further comprise delivering at least one other fetal hemoglobin inducer to the subject.

### BRIEF DESCRIPTIONS OF THE DRAWINGS

FIGS. 1A-1I show that a domain-focused CRISPR-Cas9 screen identified ZNF410 as a novel γ-globin repressor. FIG. 1A: Schematic of screening strategy. TF: transcription factor. Exp&dif: expansion and differentiation. FACS: fluorescence-activated cell sorting. FIG. 1B: Scatter plot of the screen results. Each dot represents one sgRNA. Control sgRNAs are scattered randomly across the diagonal. ZBTB7A and BCL11A represent positive control sgRNAs. FIG. 1C: Summary of HbF flow cytometric analyses. BCL11A+58: sgRNA targeting the +58 kb erythroid enhancer of the BCL11A gene serves as positive control. Non-targeting sgRNA serves as negative control. Results are shown as mean±SD (n=3). FIG. 1D: Immunoblot analysis using whole-cell lysates from differentiated HUDEP-2 cell pools transduced with indicated sgRNAs. FIG. 1E: γ-globin mRNA measured by RT-qPCR in differentiated HUDEP-2 cell pools; data are plotted as percentage of γ-globin over γ-globin+β-globin levels. Results are shown as mean±SD (n=3). FIGS. 1F-1I: mRNA levels of ε-globin (FIG. 1F), α-globin (FIG. 1G), β-globin (FIG. 1H), and GATA1 (FIG. 1I) by RT-qPCR. Results are shown as mean±SD (n=3). GAPDH was used for normalization. P values were calculated by Prism (GraphPad) with unpaired student's t-test.

FIGS. 2A-21 show that ZNF410 depletion induces γ-globin expression in primary erythroblasts. FIG. 2A: Representative flow cytometric analysis of cells stained with anti-HbF antibody on day 15 of erythroid differentiation. FIG. 2B: Summary of HbF flow cytometric analyses. Results are shown as mean±SD (n=3 donors). FIG. 2C: Immunoblot analysis using whole-cell lysates from primary erythroblasts with indicated sgRNAs on day 15 of differentiation. FIG. 2D: Representative HPLC analysis of cells with indicated sgRNAs on day 15 of differentiation. HbA: hemoglobin A (adult form); HbF: fetal hemoglobin. HbF peak area is showed as percent of total HbF+HbA. FIG. 2E: γ-globin mRNA measured by RT-qPCR in primary erythroblasts on day 12 of differentiation; data are plotted as percentage of γ-globin over γ-globin+β-globin levels. Results are shown as mean±SD (n=3 donors). FIG. 2F: Schematic of experimental design. FIGS. 2G-21: Summary of HbF flow cytometric analyses (FIG. 2G), HPLC analysis (FIG. 2H) and γ-globin mRNA measured by RT-qPCR (FIG. 2I) in human CD235a+ erythroblasts isolated from recipient bone marrows. Each dot represents a single recipient mouse. n=3 mice per sgRNA.

FIGS. 3A-3H show that CHD4 mediates γ-globin repression by ZNF410. FIG. 3A: RNA-seq analysis of HUDEP-2 cells transduced with ZNF410 sgRNA #1. Infected cells were sorted and differentiated for 7 days. Plotted is the average fold-change in mRNA levels of two biological replicates. Genes encoding NuRD complex subunits and γ-globin (HBG) are indicated. Fragments per kilobase of transcript per million (FPKM) mapped reads were used to calculate fold change. NT: non-targeting. X axis indicates the rank numbers of the genes. FIG. 3B: RNA-seq analysis of primary erythroblasts with ZNF410 depletion by sgRNA #1. Cells were differentiated for 12 days. Plotted is the average fold-change in mRNA levels of two independent donors. FIGS. 3C-3D: Immunoblot analysis using whole-cell lysates from differentiated HUDEP-2 cells (FIG. 3C) and primary erythroblasts on day 15 of differentiation (FIG. 3D). BCL11A(XL) is the functional BCL11A isoform. ImageJ software was used for the quantification. FIG. 3E: CHD4 mRNA levels measured by RT-qPCR in ZNF410 deficient HUDEP-2 cells transduced with lentiviral vector containing CHD4 cDNA or empty vector. Results are shown as mean±SD (n=2). FIG. 3F: γ-globin levels measured by RT-qPCR in ZNF410 deficient HUDEP-2 cells transduced with lentiviral vector containing CHD4 cDNA or empty vector, data are plotted as percentage of γ-globin over γ-globin+β-globin levels. Results are shown as mean±SD (n=2). FIG. 3G: Scatter plot of RNA-seq analysis in ZNF410 deficient HUDEP-2 cells (by ZNF410 sgRNA #1) with empty vector. Cells with non-targeting sgRNA and vector serve as control. Each dot indicates a gene. Each gene is depicted according to averaged FPKM value from 2 biological replicates. r: Pearson's correlation coefficient. NT: non-targeting. FIG. 3H: Scatter plot of RNA-seq analysis in ZNF410 deficient HUDEP-2 cells (by ZNF410 sgRNA #1) with re-introduction of CHD4 cDNA.

FIGS. 4A-4E show that ZNF410 binding to the CHD4 locus occurs at highly conserved motif clusters. FIG. 4A: ChIP-seq profiles of endogenous ZNF410, HA-ZNF410 and H3K27ac. CHD4 promoter and enhancer are highlighted. ZNF410 binding motifs are denoted by vertical black lines at the bottom. The 8 peak-associated genes are shown below the tracks. ZNF410 KO cells and cells transduced with empty vector serve as negative controls. HA-ZNF410: N-terminal HA tagged ZNF410. HA: hemagglutinin. FIG. 4B: Browser tracks of endogenous ZNF410 ChIP-seq occupancy at the 7 murine counterparts in differentiated mouse erythroid cells. ZNF410 binding motifs are showed at the bottom. IgG track serve as negative control. FIG. 4C: Summary of ZNF410 binding motif counts at the 8 peaks, and derived de novo motif logo in the human genome. Sequences from top to bottom are SEQ ID NO: 60, SEQ ID NO: 18, SEQ ID NO: 18, SEQ ID NO: 61, SEQ ID NO: 62, SEQ ID NO: 63, SEQ ID NO: 64, SEQ ID NO: 65, SEQ ID NO: 18, SEQ ID NO: 62, SEQ ID NO: 61, SEQ ID NO: 66, SEQ ID NO: 60, SEQ ID NO: 18, SEQ ID NO: 67, and SEQ ID NO: 18. FIGS. 4D-4E: mRNA levels of the 7 ZNF410 bound genes in HUDEP-2 cells transduced with indicated sgRNAs (FIG. 4D) and primary erythroblasts electroporated with indicated sgRNAs (FIG. 4E) by RT-qPCR (n=2).

FIGS. 5A-5H show that the ZF domain of ZNF410 is sufficient for DNA binding in vitro and in vivo. FIG. 5A: Schematic of human ZNF410. ZNF410 contains 478 amino acids, with five C2H2-type zinc fingers (ZF) between amino acids 217 and 366. FIG. 5B: ZNF410 ChIP-seq track with EMSA probes shown underneath the peaks and probe sequences showed below. Motifs are indicated. Sequences are Probe 1: SEQ ID NO: 8; Probe 2: SEQ ID NO: 9, Probe 3: SEQ ID NO: 10, and Probe 4: SEQ ID NO: 11. FIG. 5C: Full-length ZNF410 binds to the four motifs from the CHD4 promoter and enhancer sites. Left arrow: ZNF410-probe complex; right arrow: FLAG antibody-ZNF410-probe complex. *: free probes. Ab: antibody, FL: full length. FLAG-ZNF410: N-terminal FLAG tagged ZNF410. FIG. 5D: The ZF domain of ZNF410 and its truncations bind to the motif except ZF2-4. Black arrow: ZNF410 FL, ZF domain or domain truncation-probe complex. FIG. 5E: Brower tracks of endogenous ZNF410, HA ChIP-seq occupancy at the CHD4 locus in WT or HUDEP-2 cells overexpressing HA-ZF1-5 or HA-ZF2-4. FIG. 5F: mRNA levels of the 7 ZNF410 bound genes by RT-qPCR in differentiated HUDEP-2 cells with HA-ZF1-5, HA-ZF2-4 or HA-ZF410 FL overexpression. Results are shown as mean±SD (n=2). FIG. 5G: γ-globin levels measured by RT-qPCR in differentiated HUDEP-2 cells with HA-ZF1-5, HA-ZF2-4 or HA-ZF410 FL overexpression. Data are plotted as percentage of γ-globin over γ-globin+β-globin levels. Results are shown as mean±SD (n=2). FIG. 5H: The ZF domain of ZNF410 binds to the four motifs from the CHD4 promoter and enhancer sites. Ab, antibody. Bottom arrow: ZF domain-probe complex; Top arrow: FLAG antibody-ZF domain-probe complex. *: free probes.

FIGS. 6A-6K show the structural basis of ZNF410-DNA binding. FIG. 6A: Binding affinity measurements of the ZF domain against oligos by fluorescence polarization assays. Provided oligonucleotides are SEQ ID Nos: 19-22, from top to bottom. X-axis: concentration of purified GST-ZF1-5 protein, Y-axis: percentage of saturation. Pro: promoter. FIGS. 6B-6C: Two ortholog views of a ZNF410 ZF1-5 binding to DNA. FIG. 6D: Sequence alignment of the five zinc fingers of ZNF410 with DNA base-interacting positions −1, −4 and −7 in bold. The Zn-coordinating residues C2H2 of each finger are indicated. From top to bottom, the amino acid sequences are SEQ ID Nos: 68-72. FIG. 6E: General scheme of interactions between ZF1-5 and DNA. The top line indicates amino acids of each finger from C to N terminus. The first zinc-coordination His in each finger is referenced as position 0, with residues before this, at sequence positions −1, −4 and −7, corresponding to the 5′-middle-3′ of each DNA triplet element. The bottom two lines indicate the nucleotide sequence of the double-strand oligonucleotide used for crystallization (top sequence is SEQ ID NO: 19; bottom sequence is SEQ ID NO: 20). The base pair matching the consensus sequence by are numbered as 1-15. The amino acid sequences fragments of ZF 1, ZF2, ZF3, ZF4, and ZF5 are SEQ ID Nos: 74-78, respectively. FIGS. 6F-6K: Examples of base-specific contacts between each ZF and DNA. FIG. 6F: Q350 of ZF5 interacts with A3. FIG. 6G: S325 and K328 of ZF4 interacts with the C:G base pair at position 5. FIG. 6H: E322 of ZF4 interacts with C6. FIG. 6I: N295 of ZF3 interacts with A8. FIG. 6J: Q264 of ZF2 and Y238 of ZF1 interact with the T:A base pair at position 12. FIG. 6K: W232 of ZF1 interacts with A14 and T15.

FIG. 7A provides an amino acid sequence (SEQ ID NO: 1) of ZNF410. FIG. 7B provides an example of a nucleotide sequence (SEQ ID NO: 2) which encodes ZNF410. Underlined nucleotides are target sequences for sgRNA. FIG. 7C provides an amino acid sequence (SEQ ID NO: 59) of ZNF410 (isoform c). FIG. 7D provides an alignment of human (SEQ ID NO: 1) and mouse (SEQ ID NO: 73) ZNF410 protein sequences. Identical residues are identified by vertical lines. ZF (zinc finger) regions are identified and underlined.

### DETAILED DESCRIPTION OF THE INVENTION

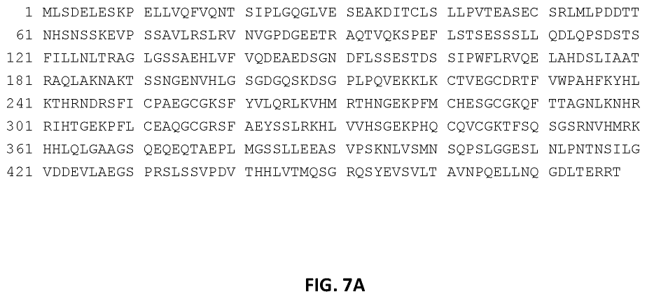

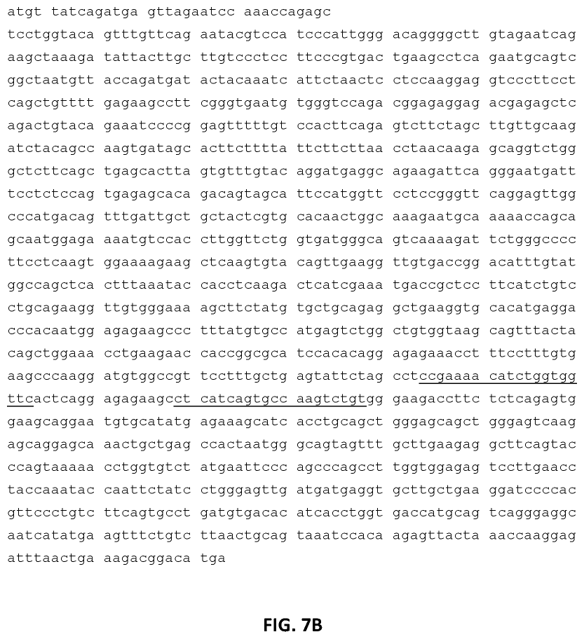

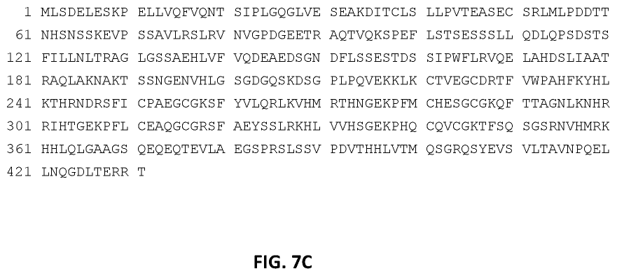

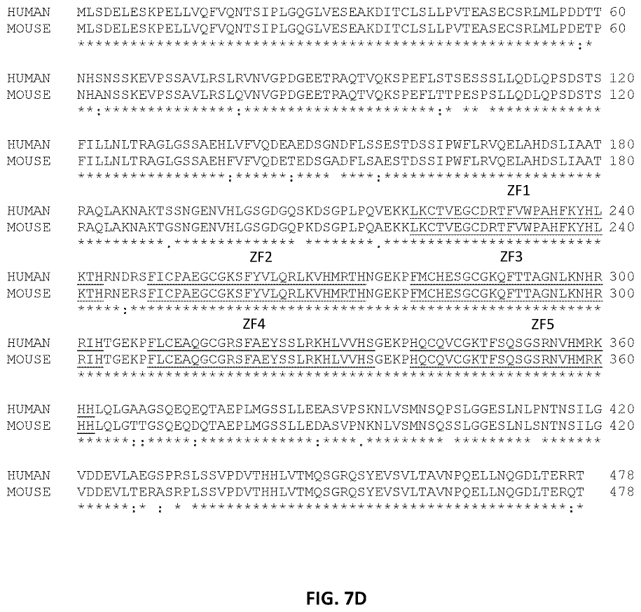

A major goal in the treatment of sickle cell disease and thalassemia is the reactivation of fetal type globin expression in cells of the adult red blood lineage. In an unbiased genetic screen, zinc finger 410 (ZNF410, APA-1) was identified as a strong repressor of fetal globin production. ZNF410 (see, e.g., PubMed GeneID: 57862) is a transcription factor with five tandem canonical C2H2-type zinc fingers (ZFs). In a particular embodiment, the ZNF410 of the instant invention is human. All splice variants and all forms, native or processed, are encompassed by the instant invention. For example, isoforms a (e.g., GenBank Accession Nos: NM_001242924.2 and NP_001229853.1), b (e.g., GenBank Accession Nos: NM_021188.3 and NP_067011.1), c (e.g., GenBank Accession Nos: NM_001242926.2 and NP_001229855.1), d (e.g., GenBank Accession Nos: NM_001242927.2 and NP_001229856.1), and e (e.g., GenBank Accession Nos: NM_001242928.2 and NP_001229857.1) of ZNF410 are encompassed by the instant invention. FIG. 7A provides an amino acid sequence (SEQ ID NO: 1) of ZNF410 (GenBank Accession NP_067011.1; isoform b). FIG. 7B provides a nucleotide sequence (SEQ ID NO: 2; GenBank Accession NM_021188.3; isoform b) which encodes ZNF410. FIG. 7C provides an amino acid sequence (SEQ ID NO: 59) of ZNF410 (GenBank Accession NP_001229855.1; isoform c).

ZNF410 may function as a transcriptional activator in human fibroblasts (Benanti, et al. (2002) Mol. Cell Biol., 22(21):7385-7397). ZNF410 may also be required for the high glucose dependent GJC1 expression in liver cancer cells (Chen, et al. (2018) J. Cell Physiol., 234(1):606-618). However, it is shown herein that the depletion of ZNF410 raises fetal hemoglobin levels. The genetic screen described herein for HbF inducers in human cells indicates that the loss of ZNF410 function increases HbF levels. Additional experiments show that the loss of ZNF410 increases fetal hemoglobin production in human erythroid cells, including primary cells. Without being bound by theory, the mechanism by which this occurs may involve in the transcriptional regulation of the known HbF regulators or co-regulators which modulate the transcriptional and/or posttranscriptional regulation of fetal hemoglobin production. This role is exploited herein to treat hemoglobinopathies such as sickle cell anemia and thalassemia.

Gene editing has emerged as a promising gene therapy for genetic diseases including β-hemoglobinopathies (Canver, et al. (2016) Blood 127(21):2536-2545). In addition, PROTAC has been recently developed to be an effective technology for targeted protein degradation (Li, et al. (2020) J. Hematol. Oncol., 13(1):50). For example, a PROTAC molecule may comprise a ligand or targeting moiety (e.g., anti-ZNF410 antibody or DNA binding domain (e.g., a nucleic acid comprising SEQ ID NO: SEQ ID NO: 18 or 19, optionally double-stranded)) for ZNF410 and a covalently linked ligand of an E3 ubiquitin ligase (E3), which will recruit E3 for ubiquitination and proteasome-mediated degradation of ZNF410. Hence, ZNF410 targeting by gene editing or PROTAC in human CD34+ hematopoietic stem and progenitor cells (HSPCs) will effectively reactivate HbF levels in these cells, which will benefit patients with sickle cell anemia or thalassemia.

In accordance with the instant invention, compositions and methods are provided for increasing hemoglobin production in a cell or subject. Compositions and methods are also provided for increasing γ-globin production in a cell or subject. In a particular embodiment, the method increases fetal hemoglobin and/or embryonic globin expression. In a particular embodiment, the method increases fetal hemoglobin.

The methods of the instant invention comprise administering at least one ZNF410 inhibitor to a cell, particularly an erythroid precursor cell or erythroid cell, or subject. In a particular embodiment, the subject has a hemoglobinopathy (such as sickle cell disease) or thalassemia. In a particular embodiment, the subject has sickle cell anemia. In a particular embodiment, the subject has thalassemia, particularly β-thalassemia, and more particularly major β-thalassemia.

The ZNF410 inhibitor may be administered in a composition further comprising at least one pharmaceutically acceptable carrier. In a particular embodiment, the method further comprises any means by which to induce fetal hemoglobin, such as administering at least one other fetal hemoglobin inducer. Fetal hemoglobin inducers include, without limitation, a lysine-specific demethylase 1 (LSD1) inhibitor (e.g., RN-1 and tranylcypromine (TCP) (Cui et al. (2015) Blood 126(3):386-96; Shi et al. (2013) Nat. Med., 19(3): 291-294; Sun et al. (2016) Reprod. Biol. Endocrinol., 14:17)), pomalidomide (Moutouh-de Parseval et al. (2008) J. Clin. Invest., 118(1):248-258; Dulmovits et al., Blood (2016) 127(11):1481-92), hydroxyurea (Charache et al., NEJM (1995) 332(20):1317-22), 5-azacytidine (Humphries et al., J. Clin. Invest. (1985) 75(2):547-57), sodium butyrate, activators or inducers of the FOXO3 pathway (e.g., metformin, phenformin, or resveratrol; Zhang et al., Blood (2018) 132(3): 321-333), L-glutamine, histone methyltransferase (HMT) inhibitors (e.g., a histone lysine methyltransferase inhibitor, euchromatic histone-lysine N-methyltransferase 2 (EHMT2; G9a) inhibitor, euchromatic histone-lysine N-methyltransferase 1 (EHMT1; G9a-like protein (GLP)) inhibitor, UNC0638 (2-cyclohexyl-N-(1-isopropylpiperidin-4-yl)-6-methoxy-7-(3-(pyrrolidin-1-yl)propoxy) quinazolin-4-amine) (Renneville et al., Blood (2015) 126(16):1930-9; Krivega et al., Blood (2015) 126(5):665-72), chaetocin, BIX-01294, UNC 0224, UNC 0642, UNC 0631, UNC 0646, A-366 (Sweis et al. (2014) ACS Med. Chem. Lett., 5(2):205-209), etc.), histone deacetylase (HDAC) inhibitors (e.g., entinostat; Bradner et al., PNAS (2010) 107(28):12617-22), and eIF2aK1 inhibitors (see, e.g., PCT/US18/15918). In a particular embodiment, the fetal hemoglobin inducer is pomalidomide or related imide, hydroxyurea, or a EHMT1/2 inhibitor such as UNC0638. In a particular embodiment, the fetal hemoglobin inducer is pomalidomide or hydroxyurea, particularly pomalidomide or similar imide. The ZNF410 inhibitor and the fetal hemoglobin inducer can be delivered to the cell or subject sequentially or consecutively (e.g., in different compositions) and/or at the same time (e.g., in the same composition).

In accordance with another aspect of the instant invention, compositions and methods for inhibiting (e.g., reducing or slowing), treating, and/or preventing a hemoglobinopathy or thalassemia in a subject are provided. In a particular embodiment, the methods comprise administering to a subject in need thereof a therapeutically effective amount of at least one ZNF410 inhibitor. The ZNF410 inhibitor may be administered in a composition further comprising at least one pharmaceutically acceptable carrier. In a particular embodiment, the hemoglobinopathy or thalassemia (e.g., β-thalassemia or sickle cell anemia). In a particular embodiment, the subject has sickle cell anemia. The methods of the instant invention may comprise administering at least two different ZNF410 inhibitors (e.g., two different mechanisms of action). In a particular embodiment, the method further comprises administering at least one other fetal hemoglobin inducer to the subject as described hereinabove. Fetal hemoglobin inducers are set forth above. In a particular embodiment, the fetal hemoglobin inducer is pomalidomide or related imide, hydroxyurea, or a EHMT1/2 inhibitor such as UNC0638. In a particular embodiment, the fetal hemoglobin inducer is pomalidomide or hydroxyurea, particularly pomalidomide. The ZNF410 inhibitor and the fetal hemoglobin inducer can be administered to the subject sequentially or consecutively (e.g., in different compositions) and/or at the same time (e.g., in the same composition).

ZNF410 inhibitors are compounds which reduce ZNF410 activity, inhibit or reduce ZNF410-substrate/partner interaction (e.g., the interaction with CHD4 (chromodomain helicase DNA binding protein 4; a key co-repressor required to silence HbF)), and/or the expression of ZNF410. The ZNF410 inhibitor may inhibit one, two, three, four, five, or all isoforms of ZNF410. In a particular embodiment, the ZNF410 inhibitor inhibits at least isoform b and/or c. In a particular embodiment, the ZNF410 inhibitor inhibits all isoforms of ZNF410.

In a particular embodiment, ZNF410 inhibitors can edit the ZNF410 gene, diminish ZNF410 expression, and/or target ZNF410 protein for degradation. In a particular embodiment, the ZNF410 inhibitor is specific to ZNF410. Examples of ZNF410 inhibitors include, without limitation, proteins, polypeptides, peptides, antibodies, small molecules, and nucleic acid molecules. In a particular embodiment, the ZNF410 inhibitor is a DNA binding domain inhibitor (e.g., a small molecule inhibitor or a nucleic acid which binds the DNA binding domain (e.g., a nucleic acid comprising SEQ ID NO: SEQ ID NO: 18 or 19, optionally double-stranded). In another embodiment, the ZNF410 inhibitor is an inhibitory nucleic acid molecule, such as an antisense, siRNA, or shRNA molecule (or a nucleic acid molecule encoding the inhibitory nucleic acid molecule). In a particular embodiment, the ZNF410 inhibitor is a small molecule. In a particular embodiment, the inhibitory nucleic acid molecule targets a sequence (e.g., is the complement of) or comprises a sequence (inclusive of RNA version of DNA molecules) as set forth in the Example provided herein (e.g., SEQ ID NO: 4 or 5). In a particular embodiment, the inhibitory nucleic acid molecule targets a sequence or comprises a sequence within the nucleic acid sequence encoding the zinc finger domains (e.g., within ZF1-ZF5). In a particular embodiment, the inhibitory nucleic acid molecule targets a sequence or comprises a sequence (e.g., RNA version) which has at least 80%, 85%, 90%, 95%, 97%, 99%, or 100% homology or identity to a sequence set forth in the Example (e.g., SEQ ID NO: 4 or 5). The sequences may be extended or shortened by 1, 2, 3, 4, or 5 nucleotides at the end of the sequence (e.g., the extended sequence may correspond to the genomic sequence). In a particular embodiment, the ZNF410 inhibitor is a CRISPR based targeting of the ZNF410 gene (e.g., with a guide RNA targeting the ZNF410 gene). In a particular embodiment, the ZNF410 inhibitor is a small molecule. The ZNF410 inhibitor may be a synthetic or non-natural compound.

In a particular embodiment, the ZNF410 inhibitor is a protein or polypeptide or a nucleic acid encoding the protein or polypeptide (e.g., an expression vector). In a particular embodiment, the ZNF410 inhibitor is the DNA-binding fragment of ZNF410. In a particular embodiment, the ZNF410 inhibitor comprises at least four ZF domains of ZNF410 (see, e.g., SEQ ID NO: 1 and FIG. 7D). For example, the ZNF410 inhibitor may comprise ZF1-ZF4, ZF2-ZF5, or ZF1-ZF5. In certain embodiments, the ZFN410 inhibitor comprises amino acids 219-334 of SEQ ID NO: 1 or amino acids 217-337 of SEQ ID NO: 1. In certain embodiments, the ZFN410 inhibitor comprises amino acids 219-362 of SEQ ID NO: 1 or amino acids 217-366 of SEQ ID NO: 1. In certain embodiments, the ZFN410 inhibitor comprises amino acids 249-362 of SEQ ID NO: 1 or amino acids 248-366 of SEQ ID NO: 1. In certain embodiments, the ZNF410 inhibitor is a fragment of ZNF410, wherein the fragment comprises 250, 225, 200, 190, 180, 175, 170, 165, 160, 155, 150 or fewer amino acids.

Clustered, regularly interspaced, short palindromic repeat (CRISPR)/Cas9 (e.g., from Streptococcus pyogenes) technology and gene editing are well known in the art (see, e.g., Sander et al. (2014) Nature Biotech., 32:347-355; Jinek et al. (2012) Science, 337:816-821; Cong et al. (2013) Science 339:819-823; Ran et al. (2013) Nature Protocols 8:2281-2308; Mali et al. (2013) Science 339:823-826; addgene.org/crispr/guide/). The RNA-guided CRISPR/Cas9 system involves expressing Cas9 along with a guide RNA molecule (gRNA). When coexpressed, gRNAs bind and recruit Cas9 to a specific genomic target sequence where it mediates a double strand DNA (dsDNA) break. The binding specificity of the CRISPR/Cas9 complex depends on two different elements. First, the binding complementarity between the targeted genomic DNA (genDNA) sequence and the complementary recognition sequence of the gRNA (e.g., −18-22 nucleotides, particularly about 20 nucleotides). Second, the presence of a protospacer-adjacent motif (PAM) juxtaposed to the genDNA/gRNA complementary region (Jinek et al. (2012) Science 337:816-821; Hsu et al. (2013) Nat. Biotech., 31:827-832; Sternberg et al. (2014) Nature 507:62-67). The PAM motif for S. Pyogenes Cas9 has been fully characterized, and is NGG or NAG (Jinek et al. (2012) Science 337:816-821; Hsu et al. (2013) Nat. Biotech., 31:827-832). Other PAMs of other Cas9 are also known (see, e.g., addgene.org/crispr/guide/#pam-table). Guidelines and computer-assisted methods for generating gRNAs are available (see, e.g, CRISPR Design Tool (crispr.mit.edu/); Hsu et al. (2013) Nat. Biotechnol. 31:827-832; addgene.org/CRISPR; and CRISPR gRNA Design tool—DNA2.0 (dna20.com/eCommerce/startCas9)). Typically, the PAM sequence is 3′ of the DNA target sequence in the genomic sequence.

In a particular embodiment, the method comprises administering at least one Cas9 (e.g., the protein and/or a nucleic acid molecule encoding Cas9) and at least one gRNA (e.g., a nucleic acid molecule encoding the gRNA) to the cell or subject. In a particular embodiment, the Cas9 is S. pyogenes Cas9. In a particular embodiment, the targeted PAM is in the 5′UTR, promoter, or first intron. When present, a second gRNA is provided which targets anywhere from the 5′UTR to the 3′UTR of the gene, particularly within the first intron. The nucleic acids of the instant invention may be administered consecutively (before or after) and/or at the same time (concurrently). The nucleic acid molecules may be administered in the same composition or in separate compositions. In a particular embodiment, the nucleic acid molecules are delivered in a single vector (e.g., a viral vector).

In a particular embodiment, the nucleic acid molecules of the instant invention are delivered (e.g., via infection, transfection, electroporation, etc.) and expressed in cells via a vector (e.g., a plasmid), particularly a viral vector. The expression vectors of the instant invention may employ a strong promoter, a constitutive promoter, and/or a regulated promoter. In a particular embodiment, the nucleic acid molecules are expressed transiently. Examples of promoters are well known in the art and include, but are not limited to, RNA polymerase II promoters, the T7 RNA polymerase promoter, and RNA polymerase III promoters (e.g., U6 and H1; see, e.g., Myslinski et al. (2001) Nucl. Acids Res., 29:2502-09). Examples of expression vectors for expressing the molecules of the invention include, without limitation, plasmids and viral vectors (e.g., adeno-associated viruses (AAVs), adenoviruses, retroviruses, and lentiviruses).

In a particular embodiment, the guide RNA of the instant invention may comprise separate nucleic acid molecules. For example, one RNA may specifically hybridize to a target sequence (crRNA) and another RNA (trans-activating crRNA (tracrRNA)) specifically hybridizes with the crRNA. In a particular embodiment, the guide RNA is a single molecule (sgRNA) which comprises a sequence which specifically hybridizes with a target sequence (crRNA; complementary sequence) and a sequence recognized by Cas9 (e.g., a tracrRNA sequence; scaffold sequence). Examples of gRNA scaffold sequences are well known in the art (e.g., 5′-GUUUUAGAGC UAGAAAUAGC AAGUUAAAAU AAGGCUAGUC CGUUAUCAAC UUGAAAAAGU GGCACCGAGU CGGUGCUUUU; SEQ ID NO: 3). As used herein, the term “specifically hybridizes” does not mean that the nucleic acid molecule needs to be 100% complementary to the target sequence. Rather, the sequence may be at least 80%, 85%, 90%, 95%, 97%, 99%, or 100% complementary to the target sequences (e.g., the complementary between the gRNA and the genomic DNA). The greater the complementarity reduces the likelihood of undesired cleavage events at other sites of the genome. In a particular embodiment, the region of complementarity (e.g., between a guide RNA and a target sequence) is at least about 10, at least about 12, at least about 15, at least about 17, at least about 20, at least about 25, at least about 30, at least about 35, or more nucleotides. In a particular embodiment, the region of complementarity (e.g., between a guide RNA and a target sequence) is about 15 to about 25 nucleotides, about 15 to about 23 nucleotides, about 16 to about 23 nucleotides, about 17 to about 21 nucleotides, about 18 to about 22 nucleotides, or about 20 nucleotides. In a particular embodiment, the guide RNA targets a sequence or comprises a sequence (inclusive of RNA version of DNA molecules) as set forth in the Example provided herein. In a particular embodiment, the guide RNA targets a sequence or comprises a sequence (e.g., RNA version) which has at least 80%, 85%, 90%, 95%, 97%, 99%, or 100% homology or identity to a sequence set forth in the Example (e.g., SEQ ID NO: 4 or 5). The sequences may be extended or shortened by 1, 2, 3, 4, or 5 nucleotides at the end of the sequence opposite from the PAM (e.g., at the 5′ end). When the sequence is extended the added nucleotides should correspond to the genomic sequence.

The above methods also encompass ex vivo methods. In certain embodiments, the methods of the instant invention use autologous cells. For example, the methods of the instant invention can comprise isolating hematopoietic cells (e.g., erythroid precursor cells) or erythroid cells from a subject, delivering at least one ZNF410 inhibitor to the cells, and administering the treated cells to the subject. The isolated cells (e.g., erythroid cells) may also be treated with other reagents in vitro, such as at least one fetal hemoglobin inducer, prior to administration to the subject.

The methods of the instant invention may further comprise monitoring the disease or disorder in the subject after administration of the composition(s) of the instant invention to monitor the efficacy of the method. For example, the subject may be monitored for characteristics of low hemoglobin or a hemoglobinopathy or thalassemia.

When an inhibitory nucleic acid molecule is delivered to a cell or subject, the inhibitory nucleic acid molecule may be administered directly or an expression vector may be used. In a particular embodiment, the inhibitory nucleic acid molecules are delivered (e.g., via infection, transfection, electroporation, etc.) and expressed in cells via a vector (e.g., a plasmid), particularly a viral vector. The expression vectors of the instant invention may employ a strong promoter, a constitutive promoter, and/or a regulated promoter. In a particular embodiment, the inhibitory nucleic acid molecules are expressed transiently. In a particular embodiment, the promoter is cell-type specific (e.g., erythroid cells). Examples of promoters are well known in the art and include, but are not limited to, RNA polymerase II promoters, the T7 RNA polymerase promoter, and RNA polymerase III promoters (e.g., U6 and H1; see, e.g., Myslinski et al. (2001) Nucl. Acids Res., 29:2502-09). Examples of expression vectors for expressing the molecules of the invention include, without limitation, plasmids and viral vectors (e.g., adeno-associated viruses (AAVs), adenoviruses, retroviruses, and lentiviruses).

As explained hereinabove, the compositions of the instant invention are useful for increasing hemoglobin production and for treating hemoglobinopathies and thalassemias. A therapeutically effective amount of the composition may be administered to a subject in need thereof. The dosages, methods, and times of administration are readily determinable by persons skilled in the art, given the teachings provided herein.

The components as described herein will generally be administered to a patient as a pharmaceutical preparation. The term “patient” or “subject” as used herein refers to human or animal subjects. The components of the instant invention may be employed therapeutically, under the guidance of a physician for the treatment of the indicated disease or disorder.

The pharmaceutical preparation comprising the components of the invention may be conveniently formulated for administration with an acceptable medium (e.g., pharmaceutically acceptable carrier) such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the agents in the chosen medium may be varied and the medium may be chosen based on the desired route of administration of the pharmaceutical preparation. Except insofar as any conventional media or agent is incompatible with the agents to be administered, its use in the pharmaceutical preparation is contemplated.

The compositions of the present invention can be administered by any suitable route, for example, by injection (e.g., for local (direct) or systemic administration), oral, pulmonary, topical, nasal or other modes of administration. The composition may be administered by any suitable means, including parenteral, intramuscular, intravenous, intraarterial, intraperitoneal, subcutaneous, topical, inhalatory, transdermal, intrapulmonary, intraarterial, intrarectal, intramuscular, and intranasal administration. In a particular embodiment, the composition is administered directly to the blood stream (e.g., intravenously). In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., polysorbate 80), anti oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington: The Science and Practice of Pharmacy, 21st edition, Philadelphia, Pa. Lippincott Williams & Wilkins. The pharmaceutical composition of the present invention can be prepared, for example, in liquid form, or can be in dried powder form (e.g., lyophilized for later reconstitution).

As used herein, “pharmaceutically acceptable carrier” includes any and all solvents, dispersion media and the like which may be appropriate for the desired route of administration of the pharmaceutical preparation, as exemplified in the preceding paragraph. The use of such media for pharmaceutically active substances is known in the art. Except insofar as any conventional media or agent is incompatible with the molecules to be administered, its use in the pharmaceutical preparation is contemplated.

Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous. Injectable suspensions may be prepared, in which case appropriate liquid carriers, suspending agents and the like may be employed. Pharmaceutical preparations for injection are known in the art. If injection is selected as a method for administering the therapy, steps should be taken to ensure that sufficient amounts of the molecules reach their target cells to exert a biological effect.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art. The appropriate dosage unit for the administration of the molecules of the instant invention may be determined by evaluating the toxicity of the molecules in animal models. Various concentrations of pharmaceutical preparations may be administered to mice, and the minimal and maximal dosages may be determined based on the results and side effects as a result of the treatment. Appropriate dosage unit may also be determined by assessing the efficacy of the treatment in combination with other standard therapies.

The pharmaceutical preparation comprising the molecules of the instant invention may be administered at appropriate intervals, for example, at least twice a day or more until the pathological symptoms are reduced or alleviated, after which the dosage may be reduced to a maintenance level. The appropriate interval in a particular case would normally depend on the condition of the patient.

### Definitions

The singular forms “a,” “an,” and “the” include plural referents unless the context clearly dictates otherwise.

The terms “isolated” is not meant to exclude artificial or synthetic mixtures with other compounds or materials, or the presence of impurities that do not interfere with the fundamental activity, and that may be present, for example, due to incomplete purification, or the addition of stabilizers.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxilliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in Remington: The Science and Practice of Pharmacy, (Lippincott, Williams and Wilkins); Liberman, et al., Eds., Pharmaceutical Dosage Forms, Marcel Decker, New York, N.Y.; and Rowe, et al., Eds., Handbook of Pharmaceutical Excipients, Pharmaceutical Pr.

The term “treat” as used herein refers to any type of treatment that imparts a benefit to a patient suffering from an injury, including improvement in the condition of the patient (e.g., in one or more symptoms), delay in the progression of the condition, etc.

As used herein, the term “prevent” refers to the prophylactic treatment of a subject who is at risk of developing a condition and/or sustaining an injury, resulting in a decrease in the probability that the subject will develop conditions associated with the hemoglobinopathy or thalassemia.

A “therapeutically effective amount” of a compound or a pharmaceutical composition refers to an amount effective to prevent, inhibit, or treat a particular injury and/or the symptoms thereof. For example, “therapeutically effective amount” may refer to an amount sufficient to modulate the pathology associated with a hemoglobinopathy or thalassemia.

As used herein, the term “subject” refers to an animal, particularly a mammal, particularly a human.

A “vector” is a genetic element, such as a plasmid, cosmid, bacmid, phage, transposon, or virus, to which another genetic sequence or element (either DNA or RNA) may be attached so as to bring about the replication and/or expression of the attached sequence or element. A vector may be either RNA or DNA and may be single or double stranded. A vector may comprise expression operons or elements such as, without limitation, transcriptional and translational control sequences, such as promoters, enhancers, translational start signals, polyadenylation signals, terminators, and the like, and which facilitate the expression of a polynucleotide or a polypeptide coding sequence in a host cell or organism.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, amino acids, or nucleic acids.

An “antibody” or “antibody molecule” is any immunoglobulin, including antibodies and fragments thereof, that binds to a specific antigen. As used herein, antibody or antibody molecule contemplates intact immunoglobulin molecules, immunologically active portions/fragment (e.g., antigen binding portion/fragment) of an immunoglobulin molecule, and fusions of immunologically active portions of an immunoglobulin molecule. Antibody fragments include, without limitation, immunoglobulin fragments including, without limitation: single domain (Dab; e.g., single variable light or heavy chain domain), Fab, Fab′, F(ab′)2, and F(v); and fusions (e.g., via a linker) of these immunoglobulin fragments including, without limitation: scFv, scFv₂, scFv-Fc, minibody, diabody, triabody, and tetrabody.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

The phrase “small, interfering RNA (siRNA)” refers to a short (typically less than 30 nucleotides long, particularly 12-30 or 20-25 nucleotides in length) double stranded RNA molecule. Typically, the siRNA modulates the expression of a gene to which the siRNA is targeted. Methods of identifying and synthesizing siRNA molecules are known in the art (see, e.g., Ausubel et al., Current Protocols in Molecular Biology, John Wiley and Sons, Inc). Short hairpin RNA molecules (shRNA) typically consist of short complementary sequences (e.g., an siRNA) separated by a small loop sequence (e.g., 6-15 nucleotides, particularly 7-10 nucleotides) wherein one of the sequences is complimentary to the gene target. shRNA molecules are typically processed into an siRNA within the cell by endonucleases. Exemplary modifications to siRNA molecules are provided in U.S. Application Publication No. 20050032733. For example, siRNA and shRNA molecules may be modified with nuclease resistant modifications (e.g., phosphorothioates, locked nucleic acids (LNA), 2′-O-methyl modifications, or morpholino linkages). Expression vectors for the expression of siRNA or shRNA molecules may employ a strong promoter which may be constitutive or regulated. Such promoters are well known in the art and include, but are not limited to, RNA polymerase II promoters, the T7 RNA polymerase promoter, and the RNA polymerase III promoters U6 and H1 (see, e.g., Myslinski et al. (2001) Nucl. Acids Res., 29:2502-09).

“Antisense nucleic acid molecules” or “antisense oligonucleotides” include nucleic acid molecules (e.g., single stranded molecules) which are targeted (complementary) to a chosen sequence (e.g., to translation initiation sites and/or splice sites) to inhibit the expression of a protein of interest. Such antisense molecules are typically between about 15 and about 50 nucleotides in length, more particularly between about 15 and about 30 nucleotides, and often span the translational start site of mRNA molecules. Antisense constructs may also be generated which contain the entire sequence of the target nucleic acid molecule in reverse orientation. Antisense oligonucleotides targeted to any known nucleotide sequence can be prepared by oligonucleotide synthesis according to standard methods. Antisense oligonucleotides may be modified as described above to comprise nuclease resistant modifications.

The following example is provided to illustrate various embodiments of the present invention. It is not intended to limit the invention in any way.

### EXAMPLE

In bacteria, one regulatory transcription factor (TF) often controls the expression of a single gene or operon (Jacob, et al. (1960) C R Hebd. Seances Acad. Sci., 250:1727-1729). In contrast, the vast majority of mammalian TFs regulate many target genes. Spatio-temporal specificity of gene transcription is achieved by combinatorial deployment of TFs and their co-regulators. For example, the transcription factor GATA1 cooperates with the TFs KLF1 and TAL1/SCL to regulate erythroid-specific gene expression (Love, et al. (2014) Trends Genet., 30:1-9) whereas GATA1 together with ETS family TFs regulates megakaryocyte-enriched genes (Wang, et al. (2002) EMBO J. 21:5225-5234). In erythroid cells, among the most highly expressed genes are those encoding the α- and β-subunits of the hemoglobin tetramer. The human β-globin gene cluster consists of one embryonic gene (HBE, also known as ε-globin), two fetal genes (HBG1 and HBG2, also known as ^{G}γ-globin and ^{A}γ-globin), and two adult genes (HBB and HBD, also known as β-globin and δ-globin) genes. The ε-globin gene is transcribed in primitive erythroid cells in early development, and during early gestation, is silenced concomitantly with the γ-globin genes turning on. Around the time of birth, a second switch occurs when β- and δ-globin transcription is activated at the expense of the γ-globin genes. Therefore, disease causing alterations in the β-globin gene such as those causing sickle cell disease (SCD) and some types of β-thalassemia become symptomatic after birth, coincident with the γ-to-β-globin switch. Reversing the switch from β-globin back to γ-globin expression in developing erythroid cells has been a major endeavor for treating these diseases (Platt, et al. (1994) N. Engl. J. Med., 330:1639-1644; Wienert, et al. (2018) Trends Genet., 34:927-940).

While lineage restricted TFs such as GATA1 and TAL1 are essential for erythroid specific transcription of the globin genes, two more widely expressed zinc-finger TFs, BCL11A and LRF (ZBTB7A) play a dominant role in the fetal-to-adult switch in globin gene transcription (Masuda, et al. (2016) Science 351:285-289; Menzel, et al. (2007) Nat. Genet., 39:1197-1199; Sankaran, et al. (2008) Science 322:1839-1842; Uda, et al. (2008) Proc. Natl. Acad. Sci., 105:1620-1625). Both of these factors bind at several locations along the β-globin gene cluster, including the promoter and upstream regions of the γ-globin genes to silence γ-globin transcription (Liu, et al. (2018) Cell 173:430-442; Martyn, et al. (2018) Nat. Genet., 50:498-503). Both factors interact with the CHD4/NuRD complex, and CHD4 and associated proteins are required for transcriptional repression of the γ-globin genes (HBG1/2) (Amaya, et al. (2013) Blood 121:3493-3501; Masuda, et al. (2016) Science 351:285-289; Sher, et al. (2019) Nat. Genet., 51:1149-1159; Xu, et al. (2013) Proc. Natl. Acad. Sci., 110:6518-6523). Given that BCL11A contains a motif found in a variety of NuRD associated molecules that is necessary and sufficient for NuRD binding (Hong, et al. (2005) EMBO J., 24:2367-2378; Lejon, et al. (2011) J. Biol. Chem., 286:1196-1203), the most parsimonious model is that BCL11A and LRF are direct links between NuRD and the γ-globin genes. One key unanswered question is whether the expression of NuRD proteins themselves is regulated, and whether control of NuRD expression might be part of the γ-globin regulatory circuitry.

To search for novel regulators of γ-globin expression, a sgRNA library targeting the DNA-binding domains of most known human transcription factors was screened using an optimized protein domain-focused CRISPR-Cas9 screening platform (Grevet, et al. (2018) Science 361:285-290; Shi, et al. (2015) Nat. Biotechnol., 33:661-667). Zinc finger 410 (ZNF410, APA-1), a transcription factor with five tandem canonical C2H2-type zinc fingers (ZFs), was found to be required for the maintenance of γ-globin silencing. RNA-seq, ChIP-seq and genetic perturbation led to the remarkable finding that ZNF410 regulates CHD4 as its sole direct target gene via two dense binding site clusters not found elsewhere in the genome. It is also demonstrated that the γ-globin genes are exquisitely sensitive to CHD4 levels. DNA binding and crystallographic studies reveal the mode of ZNF410 interaction with DNA. ZNF410 is the only known mammalian TF with a single regulatory target in erythroid cells.

Materials and Methods

The X-ray structures (coordinates and structure factor files) of ZNF410 ZF domain with bound DNA have been submitted to PDB under accession number 6WMI. The RNA-seq and ChIP-seq data have been deposited to the GEO database (GSE154963).

Cell Lines

HUDEP-2 cells were cultured and differentiated as described (Kurita, et al. (2013) PLoS One 8:e59890). Briefly, StemSpan™ Serum-Free Expansion Medium (SFEM) supplemented with 50 ng/ml human stem cell factor (SCF), 10 μM dexamethasone, 1 μg/ml doxycycline, 3 IU/ml erythropoietin and 1% penicillin/streptomycin was utilized for routine cell maintenance. Cell density was kept at 0.1-1×10⁶/ml. HUDEP-2 cells were differentiated for 6-7 days in IMDM supplemented with 50 ng/ml human SCF, 3 IU/ml erythropoietin, 2.5% fetal bovine serum, 250 μg/ml holo-transferrin, 10 ng/ml heparin, 10 μg/ml insulin, 1 μg/ml doxycycline and 1% penicillin/streptomycin.

Primary human CD34+ HSPCs from mobilized peripheral blood were purchased from the Fred Hutchinson Cancer Research Center. Human CD34+ HSPCs were differentiated using a three-phase culture system as described (Grevet, et al. (2018) Science 361:285-290). Briefly, IMDM supplemented with 3 IU/ml erythropoietin, 2.5% human male AB serum, 10 ng/ml heparin, and 10 μg/ml insulin was used as base medium. For phase I medium, 100 ng/ml human SCF, 5 ng/ml IL-3, and 250 μg/ml holo-transferrin were supplemented. For phase II medium, 100 ng/ml human SCF and 250 μg/ml holo-transferrin were added. For phase III medium, 1.25 mg/ml holo-transferrin was supplemented.

HEK293T cells were grown in DMEM supplemented with 10% fetal bovine serum, 2% penicillin/streptomycin, 1% L-glutamine and 100 μM sodium pyruvate according to standard protocol.

G1E-ER4 cells is a sub-line of G1E cells, (derived from GATA1 KO murine embryonic stem cells (Weiss, et al. (1997) Mol. Cell Biol., 17:1642-1651)), which expresses GATA1 fused to the ligand binding domain of the estrogen receptor (GATA1-ER) (Weiss, et al. (1997) Mol. Cell Biol., 17:1642-1651). GATA1 activation and erythroid differentiation are induced by the addition of 100 nM estradiol to the media for 24 hours. Cells were cultured in IMDM supplemented with 15% FBS, 1% penicillin/streptomycin, Kit ligand, monothioglycerol and erythropoietin.

COS-7 cells were cultured in DMEM supplemented with 10% fetal bovine serum and 1% penicillin-streptomycin-glutamine (PSG). For passage, adherent cells were dislodged after a 2-minute incubation at 37° C. with PBS-EDTA (5 mM).

Vector Construction

SgRNAs were cloned into a lentiviral U6-sgRNA-EFS-GFP/mCherry expression vector (LRG, Addgene: #65656) by BsmBI digestion. The ZNF410 cDNA (clone ID: OHu10535), CHD4 cDNA (clone ID: OHu28780) were purchased from GenScript and were sub-cloned into a lentiviral vector pSDM101-IRES-GFP. ZNF410 variants were sub-cloned into pSDM101-IRES-GFP vector. The N-terminal HA tag was introduced by PCR. For EMSA, the ZNF410 full length or different ZF versions were sub-cloned into mammalian expression vector pcDNA3.

Lentiviral Transduction

Lentivirus was produced as described (Grevet, et al. (2018) Science 361:285-290). Briefly, 10-20 μg of expression vectors, 5-10 μg of pVSVG (pMD2.G) and 7.5-15 μg of psPAX2 package plasmids, and 80 μl of 1 mg/ml polyethylenimine (PEI) were mixed, incubated for 15-20 minutes, and added to HEK293T cells grown in 10 cm plates to above 90% confluence. Media were replaced 6-8 hours post transfection, virus was collected 24 hours and 48 hours post-transfection and pooled. For infection, virus-containing supernatant was mixed with the indicated cell lines with 8 μg/ml polybrene and 10 mM HEPES, and then spun at 2250 rpm for 1.5 hours at room temperature. Infected HUDEP-2 cells were selected for mCherry+ or GFP+ cell sorting at 48 hours post-infection.

RNP Electroporation

Commercial sgRNAs were purchased from IDT (Coralville, Iowa) or Synthego (Menlo Park, Calif.). To assemble the RNP complexes, 100 pmol sgRNA and 50 pmol SpCas9 protein (from IDT) were incubated at room temperature for 15 minutes. CD34+ HSPCs (50k-100k) at Day 3-4 of phase I culture were electroporated using P3 Primary Cell 4D Nucleofector™ X Kit (from Lonza) with the program DZ100 (Bak, et al. (2018) Nat. Protoc., 13:358-376).

| sgRNA name | Sequence |
|---|---|
| ZNF410 sgRNA#1 | GAACCACCAGATGTTTTCGG |
| | (SEQ ID NO: 4) |
| ZNF410 sgRNA#2 | CTCATCAGTGCCAAGTCTGT |
| | (SEQ ID NO: 5) |
| BCL11A + 58 sgRNA | CTAACAGTTGCTTTTATCAC |
| | (SEQ ID NO: 6) |
| Non-targeting sgRNA | GACCGGAACGATCTCGCGTA |
| | (SEQ ID NO: 7) |

RT-qPCR

Total RNA was purified using the RNeasy® Plus Mini Kit (Qiagen), including an on-column DNAse treatment using RNase-free DNase set (Qiagen) to remove genomic DNA. Reverse transcription was accomplished using iScript™ Supermix (Bio-Rad). qPCR reactions were prepared with Power SYBR® Green (ThermoFisher Scientific). Quantification was performed using the ΔΔC_{T} method. Primers used for RT-qPCR are listed in Table 1.

TABLE 1
Primers for RT-qPCR. Provided sequences are
SEQ ID NOs: 23-58, from top to bottom.

| Name | Sequence 5′-3′ |
|---|---|
| Gamma_globin_F | TGGCAAGAAGGTGCTGACTTC |
| Gamma_globin_R | GCAAAGGTGCCCTTGAGATC |
| Beta_globin_F | TGGGCAACCCTAAGGTGAAG |
| Beta_globin_R | GTGAGCCAGGCCATCACTAAA |
| Alpha_globin_F | AAGACCTACTTCCCGCACTTC |
| Alpha_globin_R | GTTGGGCATGTCGTCCAC |
| Gamma_globin_PT_F | TTTGTGGCACCTTCTGACTG |
| Gamma_globin_PT R | GCCAAAGCTGTCAAAGAA |
| MBD2_F | AAGTGCTGGCAAGAGCGATGTCTA |
| MBD2_R | TTTCCCAGGTACCTTGCCAACTGA |
| GAPDH_F | AGCCACATCGCTCAGACAC |
| GAPDH_R | GCCCAATACGACCAAATCC |
| CHD4_F | GCTGCAACCATCCATACCTC |
| CHD4_R | ACCATCGATGCGTTCGTATT |
| GATA1_F | CTGTCCCCAATAGTGCTTATGG |
| GATA1_R | GAATAGGCTGCTGAATTGAGGG |
| MTA2_F | TTCCCACCTACACTAAGCC |
| MTA2_R | AGGCCCTTCTGAAATCCAG |
| BCL11A_F | ACAAACGGAAACAATGCAATGG |
| BCL11A_R | TTTCATCTCGATTGGTGAAGGG |
| HDAC2_F | CATGGTGATGGTGTTGAAGAAG |
| HDAC2_F | TCATTGGAAAATTGACAGCATAGT |
| GATAD2A_F | ACGAGTTCATCTACCTGGTCGG |
| GATAD2A_R | ACGTGAAGTCCGTCTTGCACTG |
| NBPF11_F | GCATGGCTGTTGACATAGGCAG |
| NBPF11_R | CTATCCAGTGAGTCCTGCAAGAC |
| LIN54_F | GTCCGACTTGTTACTGCCACATC |
| LIN54_R | TTGGAACTGACATCCGCACTGG |
| TIMELESS_F | AAGTGGTCCGGTGTCGGAGAA |
| TIMELESS_R | GTGGGCACTATTCTGCTGGTAG |
| ICAM2_F | ATGACACGGTCCTCCAATGCCA |
| ICAM2_R | GCACTCAATGGTGAAGGACTTGC |
| CBX8_F | AACATCCTGGATGCTCGCTTGC |
| CBX8_R | TTTGAGGAGGAAGGTTTTGGGCT |
| SUPT16H_F | CATTGGTGACACAGTGCTTGTGG |
| SUPT16H_R | CCAAAAGGTCCTCTGCCTCATC |

COS Cell Transfections and Nuclear Extractions

Nuclear extracts were prepared from COS-7 cells transiently transfected with ZNF410 full-length and ZNF410 ZF1-5 plasmids. FuGENE® 6 (Promega) was used to transfect 5 μg of vector into 100 mm plates of COS-7 cells. A pcDNA3 empty vector was used as control. Cells were harvested 48 hours after transfection and nuclear extracts prepared (Andrews, et al. (1991) Nucleic Acids Res., 19:2499).

In Vivo Transplantation of CD34+ HSPCs

Xenotransplantation experiments were performed (Metais, et al. (2019) Blood Adv., 3:3379-3392). Briefly, ZNF410 edited or control CD34+ HSPCs were administered at a dose of 0.4 million per NBSGW mouse (The Jackson Laboratory) by tail-vain injection at aged 8-12 weeks. Chimerism post-transplantation was assessed by flow analysis at 8 weeks in the periphery and at 16 weeks in the bone marrow at the time of euthanasia. Cell linage composition was determined in the bone marrow using human-specific antibodies, and different lineages were sorted by a FACSAria™ III cell sorter. CD34+ HSPCs were isolated with magnetic beads using the human-specific CD34 MicroBead Kit UltraPure, human (Miltenyi Biotec Inc).

Indel Analysis

Next-generation sequencing (NGS) was used for indel analysis (Metais, et al. (2019) Blood Adv., 3:3379-3392). Briefly, NGS libraries were prepared with a 2-step PCR protocol. In the first step, the targeted genomic sites were amplified by PCR with Phusion® Hot Start Flew 2x Master Mix (New England BioLabs) and primers with partial Illumina sequencing adaptors. In the second step, PCR was performed with a KAPA HiFi HotStart® ReadyMix PCR Kit (Roche) to add Illumina sequencing adapters (P5-dual-index and P7-dual-index) to the purified PCR product from the first step. The Illumina MiSeg™ platform was used to generate FASTQ sequences with 150 bp paired-end reads, and these reads were analyzed by joining paired reads and analyzing amplicons, using CRISPResso for indel measurement.

Primers for NGS sequencing:
ZNF410 sgRNA#1:
Fwd:
(SEQ ID NO: 8)
GCCTCATATCCCATAATATTCAGCCCCAT
Rev:
(SEQ ID NO: 9)
GAGCCAGGCATCCCATAATATTCATATTCT
ZNF410 sgRNA#2:
Fwd:
(SEQ ID NO: 10)
ACACACGACATCCCATAATATCTTCTGGAG
Rev:
(SEQ ID NO: 11)
GCCTCACCAACCCATAATATTCCCCAGTCT.

EMSAs

EMSAs were performed (Crossley, et al. (1996) Mol. Cell Biol., 16:1695-1705). The sense oligonucleotide was labelled with [γ-³²P]-adenosine triphosphate (Perkin Elmer) and boiled at 100° C. for 1 minute before addition of the antisense oligonucleotide and annealing of probe via slow cooling from 100° C. to room temperature. Probes were purified using Quick Spin Columns for radio-labelled DNA Purification (Roche). Nuclear extracts were harvested from COS-7 cells and samples and loaded on a 6% native polyacrylamide gel in TBE buffer (45 mM Tris, 45 mM boric acid, 1 mM EDTA). A ‘COS empty’ control lane was included to show binding of any background endogenous protein to the probe. Recognition and super-shifting of FLAG-ZNF410 overexpression constructs was achieved with an anti-FLAG monoclonal antibody (Sigma). Gels were run at 250 V for 1 hour, 45 minutes at 4° C. then dried under vacuum. Gels were exposed overnight with a FUJIFILM BAS CASETTE2 2025 phosphor screen and imaged using the Typhoon™ FLA 9500 Laser Scanner.

HbF Staining and Flow Cytometry

Briefly, 2-5 million cells were fixed in 0.05% glutaraldehyde for 10 minutes, washed 3 times with 1×PBS/0.1% BSA, and permeabilized with 0.1% Triton X-100 for 5 minutes. After one wash with PBS/0.1% BSA, cells were stained with HbF-APC conjugate antibody for 15-30 minutes in the dark at room temperature. Cells were washed twice with PBS/0.1% BSA. Flow cytometry was carried out on a BD FACSCanto™ and cell sorting on a BD FACSJazz™ at the Children's Hospital of Philadelphia flow cytometry core.

CRISPR sgRNA Library Generation and Screen

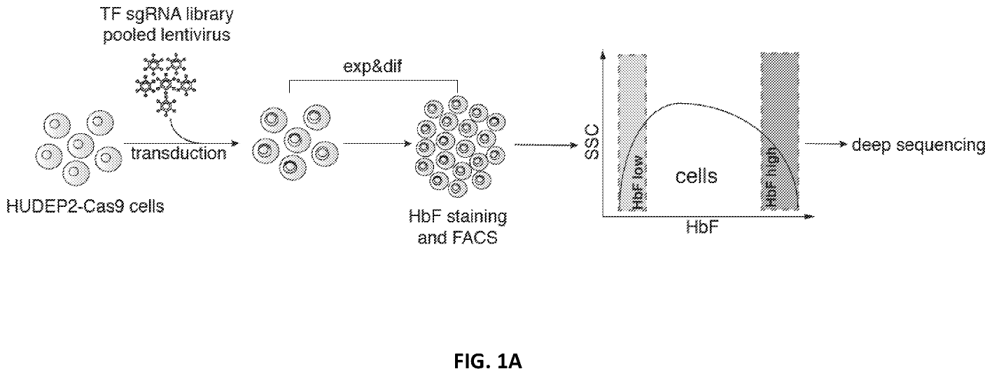

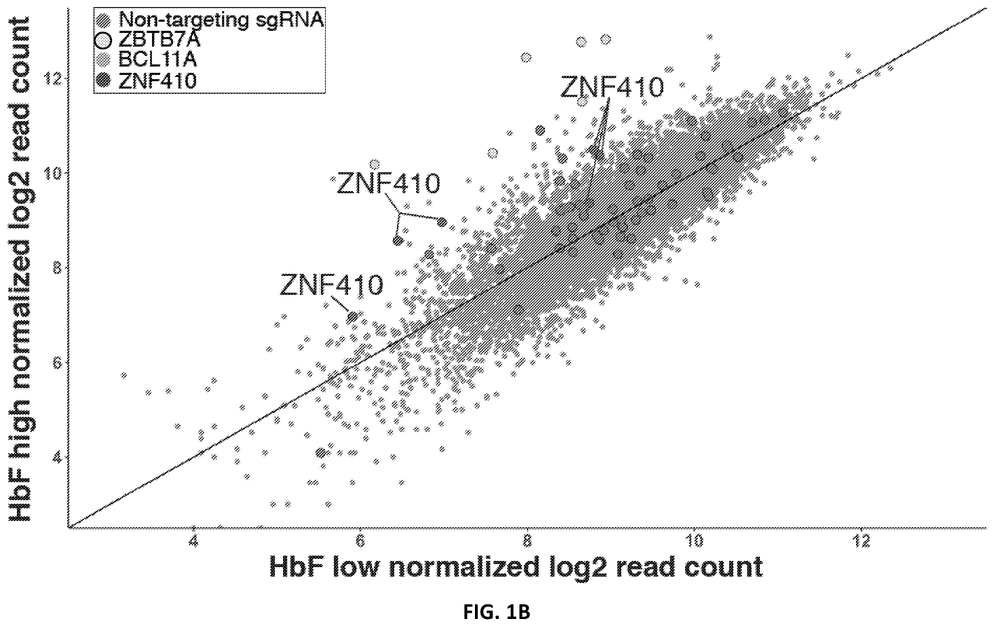

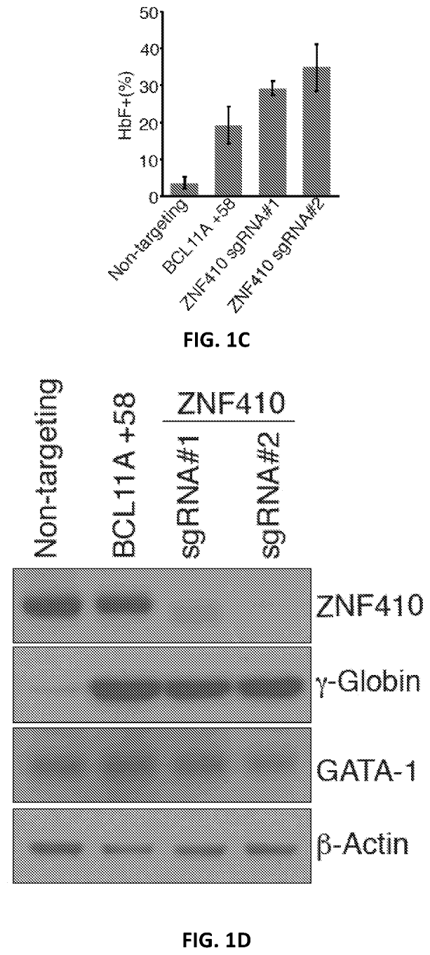

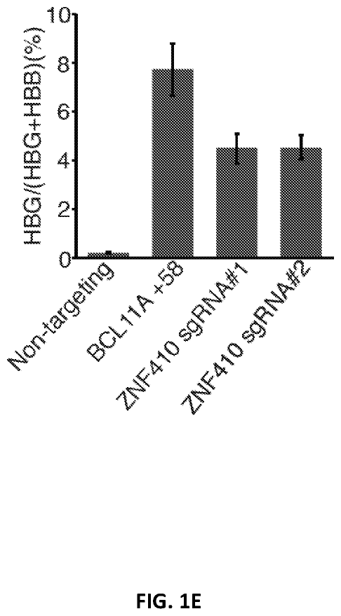

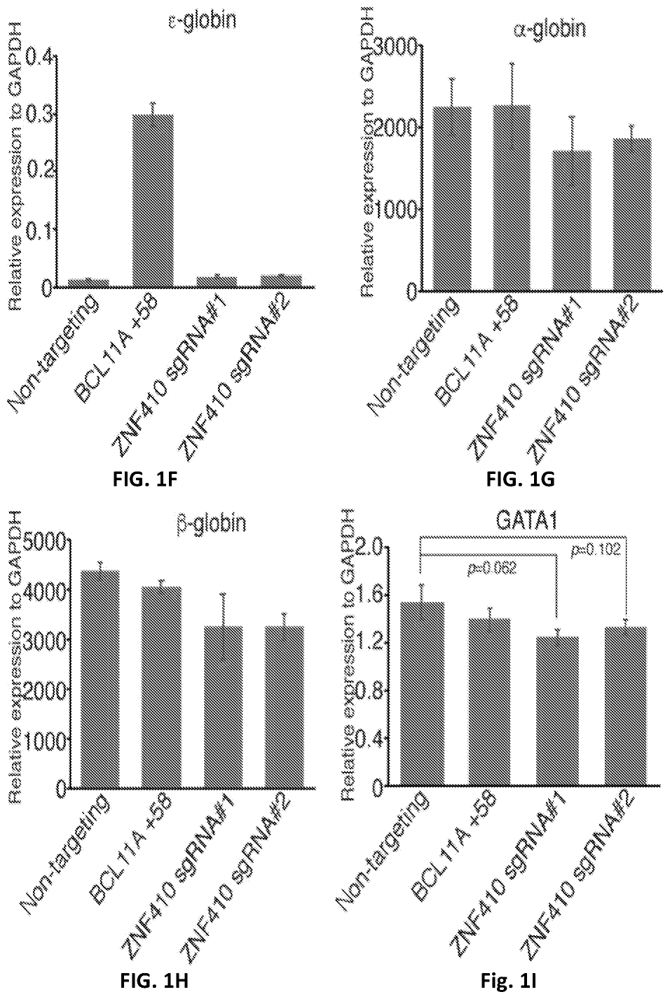

SgRNA library targeting human transcription factors and the screening protocol were performed as described previously (Huang, et al. (2020) Blood 135:2121-2132; Grevet, et al. (2018) Science 361:285-290). Briefly, HUDEP2-Cas9 cells were transduced with the transcription factor library at a low multiplicity of infection (MOI 0.3-0.5). ˜30 million cells were infected in total to yield 1000× coverage of the sgRNA library in the GFP+ population. Transduced cells were sorted by GFP+ FACS on day 2 post-infection. Transduced cells were cultured in HUDEP2 media for an additional 6 days (total 8 days post-infection). On day 8 post-infection, cells were switched to differentiation media and cultured for 7 days. On day 15 post-infection, cells were stained for HbF, and sorted into HbF high and HbF low populations (see FIG. 1A).

Genomic DNA was extracted from these samples by phenol/chloroform extractions per standard methods. sgRNAs were amplified with Phusion™ Flash High Fidelity Master Mix Polymerase per manufacturer specifications. PCR reactions were then pooled for each sample and column purified with QIAGEN PCR purification kit. PCR products were subjected to Illumina MiSeg™ library construction and sequencing. sgRNA library concentrations were quantified on a 2100 Bioanalyzer (Agilent). The barcoded libraries were pooled at an equal molar ratio and subjected to massively parallel sequencing through a MiSeg™ instrument (Illumina) using 75-bp paired-end sequencing (MiSeq™ Reagent Kit v3; Illumina MS-102-3001).

The sequencing data were de-barcoded and trimmed to contain only the sgRNA sequence, and subsequently mapped to the reference sgRNA library without allowing any mismatches. The read counts were calculated for each individual sgRNA and normalized to total read counts. Normalized read counts of sgRNAs in HbF high and HbF low populations were log₂ transformed in RStudio software.

Immunoblot Analysis

Cells were lysed in RIPA buffer containing protease inhibitors (Sigma) and PMSF for 20-30 minutes on ice. Cell lysates were mixed with 5× Lammli sample buffer, and then boiled at 95 degrees for 5-10 minutes. ˜15-30 μg whole cell lysates per sample were loaded on NuPAGE™ 4-12% Bis-Tris protein Gels (Thermofisher). After transfer, nitrocellulose membrane was first blocked by 5% nonfat milk in TBST, and incubated with primary antibody in 5% milk at 4° C. overnight. Membranes were washed 3 times with 1×TBST, followed by incubation with secondary antibody for 1 hour at room temperature, and then incubated with chemiluminescent HRP substrate (Thermofisher).

RNA-Seq

Total RNAs were purified as described above. Sequencing libraries were then constructed using 100 ng of purified total RNA using the ScriptSeg™ Complete Kit (Illumina cat #BHMR1224) according to manufacturer's protocol. RNA was subjected to rRNA depletion using the Ribo-Zero™ removal reagents and fragmented. First strand cDNA was synthesized using a 5′ tagged random hexamer, and reversely transcribed, followed by annealing of a 5′ tagged, 3′-end blocked terminal-tagged oligo for second strand synthesis. The Di-tagged cDNA fragments were purified, barcoded, and PCR-amplified for 15 cycles.

The size and quality of each library were evaluated by Bioanalyzer 2100 (Agilent Technologies, Santa Clara, Calif.), and quantified using qPCR. Libraries were sequenced in paired-end mode on a NextSeq 500 instrument to generate 2×76 bp reads using Illumina-supplied kits. The sequence reads were processed using the ENCODE3 long RNA-seq pipeline (encodeproject.org/pipelines/ENCPL002LPE/). In brief, reads were mapped to the human genome (hg38 assembly) using STAR, followed by RSEM for gene quantifications.

RNA-Seq Data Analysis

The normalized FPKM (fragments per kilo base per million mapped reads) for each gene was averaged in 2 replicates and then filtered to keep those with average FPKM at least 10 in both HUDEP-2 cells and primary erythroblasts, resulting in ˜5000 high abundant genes each cell type for further analysis. Log₂ fold-change was calculated from FPKM of sgRNA targeting ZNF410 compared to control sgRNA (non-targeting sgRNA) using the DESeq2 method, and top changed genes were selected with fold-change at least 1.5 and p-value<0.05. Commonly changed genes in both independent sgRNAs were considered to be significant. Scatter plots were generated using ggplot2 in RStudio for all expressed genes (FPKM>5).

ChIP-Seq

HUDEP-2 cells at Day 3 of differentiation, primary human CD34+ cells at Day 9 differentiation (similar to the polychromatic stage) and G1E-ER4 cells at 24 hours differentiation were crosslinked with 1% formaldehyde at room temperature for 10 min and quenched by the addition of glycine. ChIP experiments were performed (Hsu, et al. (2017) Mol. Cell., 66:102-116). ZNF410 (Proteintech, Cat. #14529-1-AP), HA (Sigma, Cat. #11815016001) and H3K27ac (Abcam, Cat. #ab4729) antibodies were used for ChIP. ChIP-seq libraries were prepared using TruSeq® ChIP-seq Sample preparation Kit (part #IP-202-1012) according to the manufacturer's instructions. Reads were aligned with Bowtie2 local alignment to allow the mapping of indels (Langmead, et al. (2012) Nat. Methods 9:357-359). All ChIP-seq experiments were performed in two biological replicates. ChIP-qPCR was performed with Power SYBR® Green (ThermoFisher). Primers for ChIP-qPCR:

CHD4 promoter:
Fwd:
(SEQ ID NO: 12)
GCAGACCTTTTGCAACTAACC
Rev:
(SEQ ID NO: 13)
GGGGTGCTTATTATGGGATG
CHD4 enhancer:
Fwd:
(SEQ ID NO: 14)
AGCAGCCATCCCATAATAGC
Rev:
(SEQ ID NO: 15)
CTCCATTTCCTCTCCAGCTC
HBG2:
Fwd:
(SEQ ID NO: 16)
TCACACACACAAACACACG
Rev:
(SEQ ID NO: 17)
AGATGGGGGCAAAGTATGTC.

ZNF410 ChIP-Peak Calling and De Novo Motif Analysis

Reads were aligned against reference genome hg38 for human and genome mm10 for mouse using Bowtie2 (v2.2.9) and the default parameters. Alignments with MAPQ score lower than 10 and PCR duplicates were removed using Samtools (v0.1.19). Reads aligned to mitochondria, random contigs and ENCODE blacklisted regions were also removed for downstream analysis. Genome coverage files were generated and normalized to 1 million reads per library using bedtools (v2.25.0), and then converted to bigwig format for visualization using the UCSC Toolkit. Peaks were called using MACS2 (v2.1.0) and a 0.05 q-value cutoff. The final peaks were those overlapped by both ZNF410 replicates but not in control replicates (empty vector and knock-out samples), then manually filtered to exclude peaks near centromere/telomere regions that did not look like peaks on genome browser (total number reduced from 38 to 8). The final peaks were extended by 1 kb on both ends for de novo motif analysis using the HOMER tool, and the top hit motif was scanned across the entire genome using HOMER. The human and mouse genomes were also scanned for motif pattern of CATCCCATAATA (SEQ ID NO: 18) and other similar motifs using EMBOSS fuzznuc (v6.5.7.0). Read density plot and heatmap around selected peaks were generated using Deeptools (version 2.5.7, “computeMatrix” and “plotHeatmap”).

HPLC

˜1 million primary erythroblasts (at the orthochromatic stage) were lysed in water for 10 minutes, vertex 10 seconds every 5 minutes at room temperature. Hemolysates were then cleared by centrifugation at 15,000 rpm, 10 minutes and analyzed for identity and levels of hemoglobin variants (HbF and HbA) by cation-exchange high-performance liquid chromatography (HPLC). Hitachi D-7000 Series (Hitachi Instruments, Inc., San Jose, Calif.), and weak cation-exchange column (Poly CAT A: 35 mm×4.6 mm, Poly LC, Inc., Columbia, Md.) were used. Hemoglobin isotype peaks were eluted with a linear gradient of phase B from 0% to 80% at A_{410 nm} (Mobile Phase A: 20 mM Bis-Tris, 2 mM KCN, pH 6.95; Phase B:20 mM Bis-Tris, 2 mM KCN, 0.2 M sodium chloride, pH 6.55). Cleared lysates from normal human cord blood samples (high HbF content), as well as a commercial standard containing approximately equal amounts of HbF, A, S and C (Helena Laboratories, Beaumont, Tex.), were utilized as reference isotypes.

Wright-Giemsa Staining

˜100,000 cells were spun onto glass slides with Cytospin® 4 (ThermoFisher Scientific) at 1,200 rpm for 3 minutes. Slides were allowed to dry for 5 minutes at RT, followed by staining with May Grunwald (Sigma Aldrich) for 2 minutes and then by 1:20 diluted Giemsa stain (Sigma Aldrich) for 10 minutes. The stained slides were rinsed twice in water and then allowed to dry for 10 minutes before a coverslip was sealed on the preparation with Cytoseal™ 60 (Thermo Scientific). The images were captured with Olympus BX60 microscope at 10× resolution using Infinity software (Lumenera corporation).

Protein Expression and Purification

The fragment of Human ZNF410 (NP_001229855.1) comprising of five zinc finger domains ZF1-5 (residues 217-366) was cloned into pGEX-6P-1 vector with a GST fusion tag (pXC2180). The plasmid was transformed into Escherichia coli strain BL21-Codon-plus(DE3)-RIL (Stratagene). Bacteria was grown in LB broth in a shaker at 37° C. until reaching the log phase (A_{600 nm} between 0.4 and 0.5), the shaker temperature was then set to 16° C. and 25 μM ZnCl₂ was added to the cell culture. When the shaker temperature reached 16° C. and A_{600 nm} reached ˜0.8, the protein expression was induced by the addition of 0.2 mM isopropyl-3-D-thiogalactopyranoside with subsequent growth for 20 hours at 16° C. Cell harvesting and protein purification were carried out at 4° C. through a three-column chromatography protocol (Patel, et al. (2016) Methods Enzymol., 573:387-401), conducted in a BIO-RAD NGC™ system. Cells were collected by centrifugation and pellet was suspended in the lysis buffer consisting of 20 mM Tris-HCl, pH 7.5, 500 mM NaCl, 5% glycerol, 0.5 mM tris(2-carboxyethl)phosphine (TCEP) and 25 μM ZnCl₂. Cells were lysed by sonication and 0.3% (w/v) polyethylenimine was slowly titrated into the cell lysate before centrifugation (Patel, et al. (2016) Methods Enzymol., 573:387-401). Cell debris was removed by centrifugation for 30 minutes at 47,000×g and the supernatant was loaded onto a 5 ml GSTrap™ column (GE Healthcare). The resin was washed by the lysis buffer and bound protein was eluted with elution buffer of 100 mM Tris-HCl, pH 8.0, 500 mM NaCl, 5% glycerol, 0.5 mM TCEP and 20 mM reduced form glutathione. The GST fusion were digested with PreScission™ protease to remove the GST fusion tag. The cleaved protein was loaded onto a 5 ml Heparin column (GE Healthcare). The protein was eluted by a NaCl gradient from 0.25 to 1 M in 20 mM Tris-HCl, pH 7.5, 5% glycerol and 0.5 mM TCEP. The peak fractions were pooled, concentrated and loaded onto a HiLoad® 16/60 Superdex® 5200 column (GE Healthcare) equilibrated with 20 mM Tris-HCl, pH 7.5, 250 mM NaCl, 5% glycerol and 0.5 mM TCEP. The protein was frozen and stored at −80° C.

DNA Binding Assays

Fluorescence polarization (FP) method was used to measure the binding affinity using a Synergy™ 4 Microplate Reader (BioTek). Aliquots (5 nM) of 6-carboxy-fluorescein (FAM)-labeled DNA duplex (FAM-5′-CACA TCC CAT AAT AATG-3′ (SEQ ID NO: 19) and 3′-GTGT AGG GTA TTA TTAC-5′ (SEQ ID NO: 20)) and control (FAM-5′-TCC ACT GCC AGG ACC TTT-3′ (SEQ ID NO: 21) and 3′-GGT GAC GGT CCT GGA AAA-5′ (SEQ ID NO: 22)) was incubated with varied amount of proteins (0 to 2.5 μM) in 20 mM Tris-HCl, pH 7.5, 300 mM NaCl, 5% glycerol and 0.5 mM TCEP for 10 minutes at room temperature. The data were processed using Graphpad Prism (version 8.0) with equation [mP]=[maximum mP] ×[C]/(KD+[C])+[baseline mP], in which mP is millipolarization and [C] is protein concentration. The KD value for each protein-DNA interaction was derived from two replicated experiments.

Electrophoretic mobility shift assay (EMSA) was performed with the same set of samples used in the FP assay for 10 min at room temperature. Aliquots of 10 μl of reactions were loaded onto an 8% native 1×TBE polyacrylamide gel and run at 150V for 20 minutes in 0.5×TBE buffer. The gel was imaged using a ChemiDoc™ Imaging System (BIO-RAD).

Crystallography

The ZF-DNA complex was prepared by mixing 0.9 mM ZF1-5 fragment and double-stranded DNA oligo (annealed in buffer containing 10 mM Tris-HCl, pH 7.5, and 50 mM NaCl) with molar ratio 1:1.2 of protein to DNA on ice for 30 minutes incubation. The protein-DNA complex crystals were grown using the sitting drop vapor diffusion method via an Art Robbins Gryphon Crystallization Robot at 19° C. with a well solution of 0.2 M ammonium formate and 20% polyethylene glycol 3350. Crystals were flash frozen using 20% (v/v) ethylene glycol as the cryo-protectant. The X-ray diffraction data were collected at SER-CAT 22-ID beamline of the Advanced Photon Source at Argonne National Laboratory utilizing a X-ray beam at 1.0 Å wavelength and processed by HKL2000 keeping Friedel mates separate (Otwinowski, et al. (2003) Acta Crystallogr. A, 59:228-234).

The resultant dataset for ab initio phasing was examined using the PHENIX Xtriage module (Adams, et al. (2002) Acta Crystallogr. D Biol. Crystallogr., 58:1948-1954) which reported a very good anomalous signal to 5.6 Å. The PHENIX AutoSol module (Terwilliger, et al. (2009) Acta Crystallogr. D Biol. Crystallogr., 65:582-601) identified the space group being P6₂ and found all 10 zinc atom positions (5 per each of two molecules in asymmetric unit) with a Figure-Of-Merit of 0.48 and gave a density modified map with an R-factor of 0.34 at 5 Å data. Insertion of these zinc positions into AutoSol and utilizing the full resolution of the dataset gave a Figure-Of-Merit of 0.28 and a density modified map with an R-factor of 0.34. DNA duplex and zinc fingers bound in the major groove could easily be identified for the resultant map. The AutoBuild module of PHENIX was utilized for model building, and manual fitting of the protein and the DNA duplex was completed with COOT (Emsley, et al. (2004) Acta Crystallogr. D Biol. Crystallogr., 60:2126-2132), which was also utilized for corrections between PHENIX refinement rounds. Structure quality was analyzed during PHENIX refinements and finally validated by the PDB validation server. Molecular graphics were generated by using PyMol (Schrödinger, LLC).

Quantification and Statistical Analysis

ImageJ software was used for quantification of immunoblots. Statistical significance was evaluated by p-value from unpaired Student t-test using Prism software.

Results

CRISPR-Cas9 Screen Identifies ZNF410 as a Candidate γ-Globin Repressor

To identify novel regulators of HbF expression, a sgRNA library containing 6 sgRNAs each targeting the DNA-binding domain of most human transcription factors was screened (1436 total, on average 6 sgRNAs each) (Huang, et al. (2020) Blood 135:2121-2132). A lentiviral vector library encoding the sgRNAs was used to transfect the human adult-type erythroid cell line HUDEP-2 that stably expresses spCas9 (HUDEP-2-Cas9; (Grevet, et al. (2018) Science 361:285-290)). The top 10% and bottom 10% of HbF expressing cells were purified via anti-HbF FACS, and representation of each sgRNA in the two populations assessed by deep sequencing (FIG. 1A). As expected, control non-targeting sgRNAs were evenly distributed between the HbF-high and HbF-low populations. Positive control sgRNAs targeting the known γ-globin repressor genes BCL11A and LRF were enriched in the HbF-high population (FIG. 1B), validating the screen. Six sgRNAs against a novel TF, ZNF410, with no known prior role in globin gene regulation were significantly enriched in the HbF-high population (FIG. 1B), indicating that ZNF410 may function as a direct or indirect repressor of γ-globin expression.

ZNF410 may function as a transcriptional activator in human fibroblasts (Benanti, et al. (2002) Mol. Cell Biol., 22:7385-7397). ZNF410 is widely expressed across human tissues (Genotype-Tissue Expression database). In blood, ZNF410 is highly expressed in the erythroid lineage (BloodSpot), and its mRNA levels are similar between fetal and adult erythroblasts (Huang, et al. (2017) Genes Dev., 31:1704-1713).

To validate the screening results, two independent sgRNAs targeting the DNA-binding domain of ZNF410 were stably introduced into HUDEP-2-Cas9 cells along with a positive control sgRNA (targeting the +58 erythroid enhancer of the BCL11A gene) and non-targeting negative control sgRNA. Depletion of ZNF410 strongly increased the fraction of HbF-expressing cells as determined by flow cytometry using anti-HbF antibodies (FIG. 1C). Western blotting revealed substantial elevation of γ-globin protein in ZNF410 depleted HUDEP-2 cells. Protein levels of GATA1 were unchanged, consistent with erythroid maturation being intact in these cells (FIG. 1D). To assess whether ZFN410 impacts the transcription of the γ-globin gene, RT-qPCR was performed. A robust increase in primary and mature γ-globin mRNA occurred upon ZFN410 depletion, indicating transcriptional regulation (FIG. 1E). ZNF410 loss did not impact ε-globin mRNA levels, indicating specificity for the fetal globin genes (FIG. 1F). Importantly, there were no significant changes in α-globin, β-globin, and GATA1 mRNA levels (FIGS. 1G-1I), indicating that ZNF410 depletion did not overtly impair erythroid differentiation, an observation further supported by RNA-seq analysis. Taken together, the screen identified ZNF410 as novel repressor of γ-globin gene expression in HUDEP-2 cells.

Depletion of ZNF410 Elevates γ-Globin Levels in Primary Human Erythroblasts

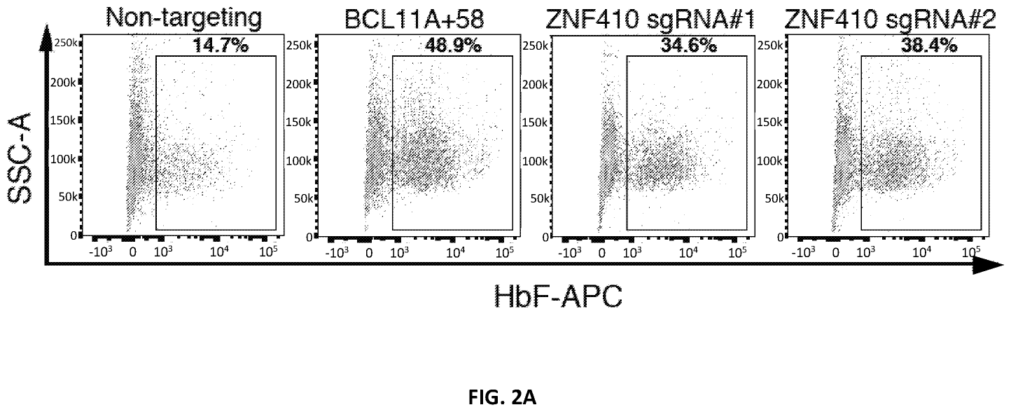

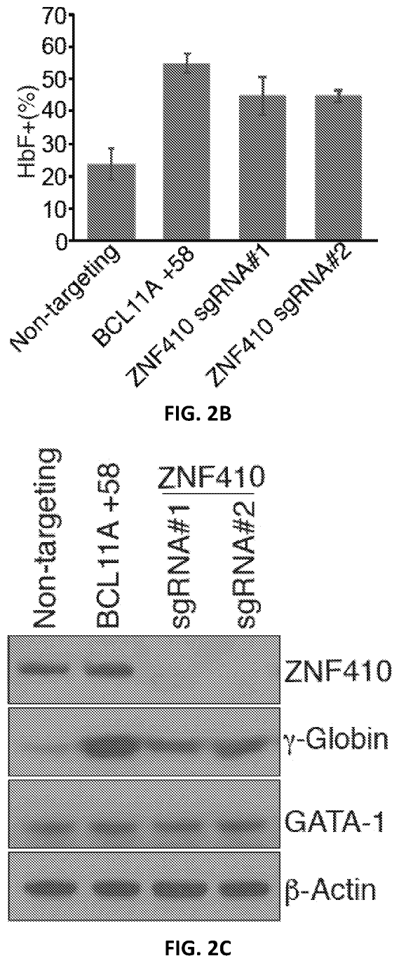

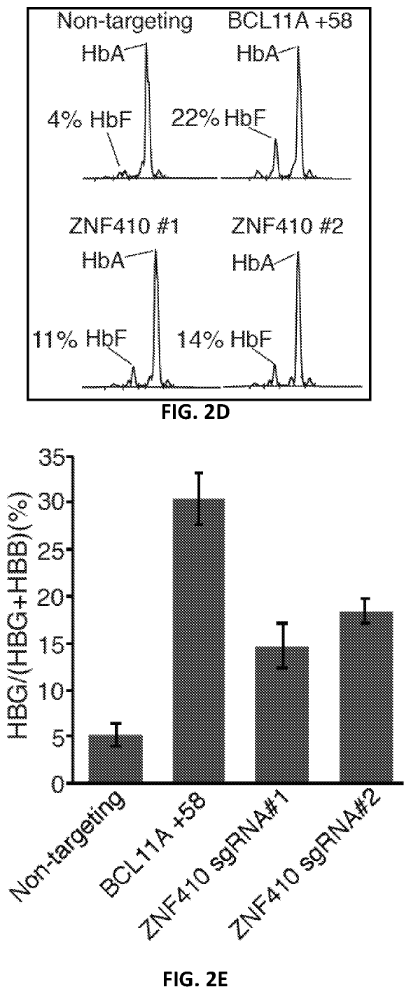

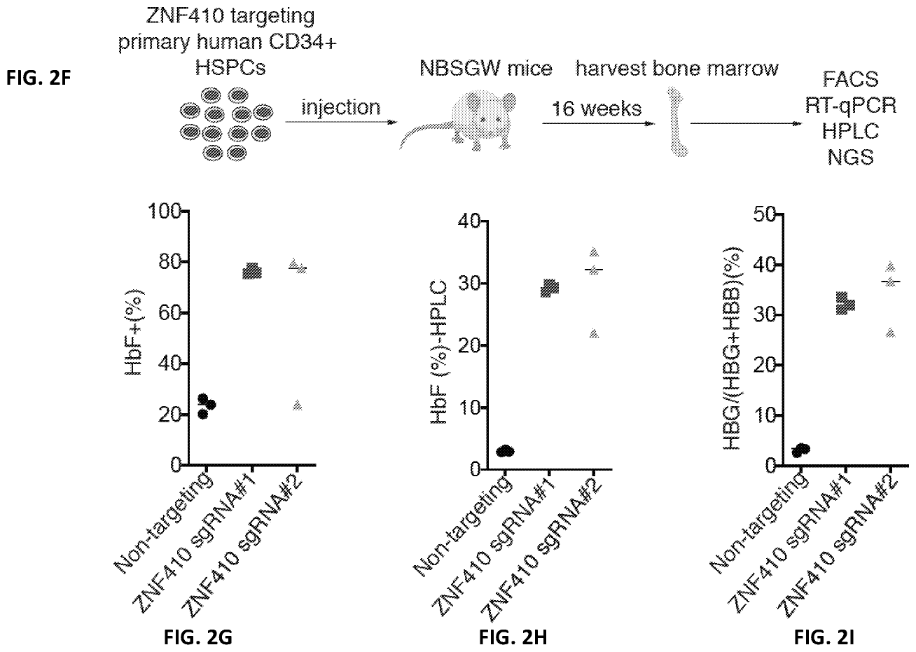

The repressive role of ZNF410 on HbF was further tested in primary human erythroblasts derived from a three-phase human CD34+ hematopoietic stem and progenitor cells (HSPCs) culture system (Grevet, et al. (2018) Science 361:285-290). ZNF410 was depleted by electroporating ribonucleoprotein (RNP) Cas9:sgRNAs complexes using two independent sgRNAs. A sgRNA targeting the erythroid +58 enhancer of BCL11A was used as positive control. In line with findings in HUDEP-2 cells, ZNF410 depletion significantly elevated the proportion of HbF+ cells (FIGS. 2A and 2B), γ-globin protein levels (Western blot, FIG. 2C), HbF protein levels (HPLC, FIG. 2D), and γ-globin primary and mature mRNA (FIG. 2E). Moreover, ε-globin mRNA levels were unaffected and α-globin, β-globin, and GATA1 levels were not significantly changed, indicating that ZNF410 loss did not adversely affect maturation of these cells. Cell surface marker phenotyping using anti-CD71 and anti-CD235a antibodies as well as examination of cell morphology indicated normal erythroid maturation in ZNF410 deficient cells.

A human-to-mouse xenotransplantation model was used to further assess the role of ZNF410 on the regulation of γ-globin in vivo. Healthy adult human donor CD34+ HSPCs were transfected with ribonucleoprotein complex consisting of spCas9+two sgRNAs (analyzed separately) or a non-targeting sgRNA as negative control, and then transplanted them into NBSGW immunodeficient mice that support human erythropoiesis in the bone marrow (McIntosh, et al. (2015) Stem Cell Reports 4:171-180). The fraction of various engrafted human lineages and their gene editing frequencies was measured in recipient bone marrow at 16 weeks after xenotransplantation (FIG. 2F), a time at which CD34+ progenitor cells are mainly derived from the human transplant (McIntosh, et al. (2015) Stem Cell Reports 4:171-180). Donor chimerism of ZNF410 edited CD45+ hematopoietic cells was slightly lower than in control cells exposed to non-targeting sgRNA. Chimerism levels and indel frequencies were similar in all edited and nonedited lineages tested, including B-cells, myeloid, erythroid and progenitor cells, indicating that ZNF410 depletion did not overtly impact hematopoietic development. Importantly, in the erythroid compartment (CD235+), a robust increase in the fraction of HbF+ cells (from −23% to −78%), HbF protein levels (from −3% to −33%), and γ-globin mRNA levels (from −3% to −32%) was observed (FIGS. 2G-21), consistent with the results in HUDEP-2 and cultured primary human erythroblasts. Again, depletion of ZNF410 did not appear to impair erythroid maturation as determined by flow cytometry analysis of erythroid maturation markers CD49d/Band3. Collectively, these in vivo studies verify that ZNF410 functions as a robust repressor of HbF with little or no detrimental effect on hematopoietic development.

ZNF410 Represses HbF by Modulating CHD4 Expression

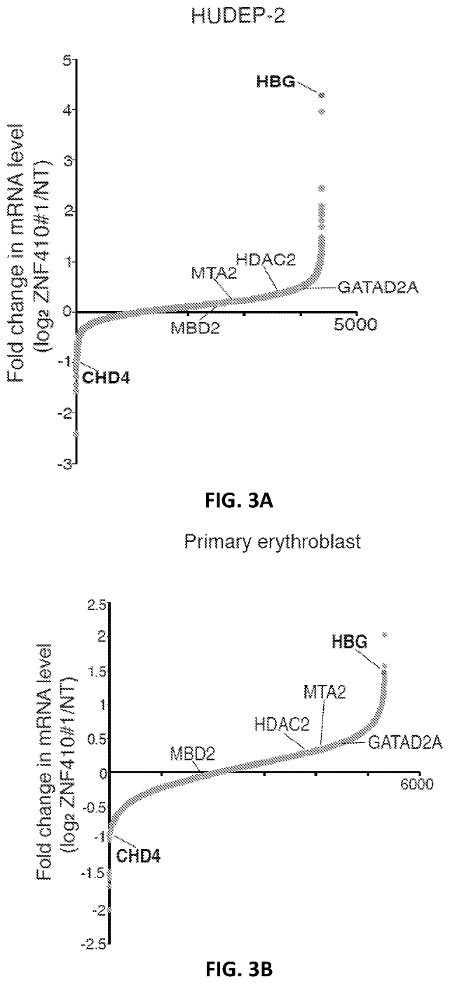

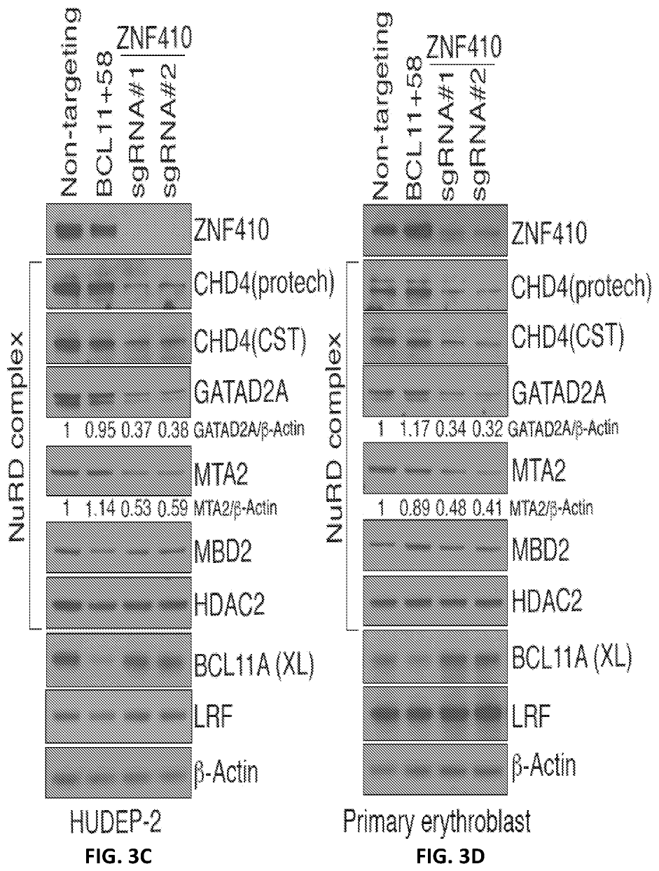

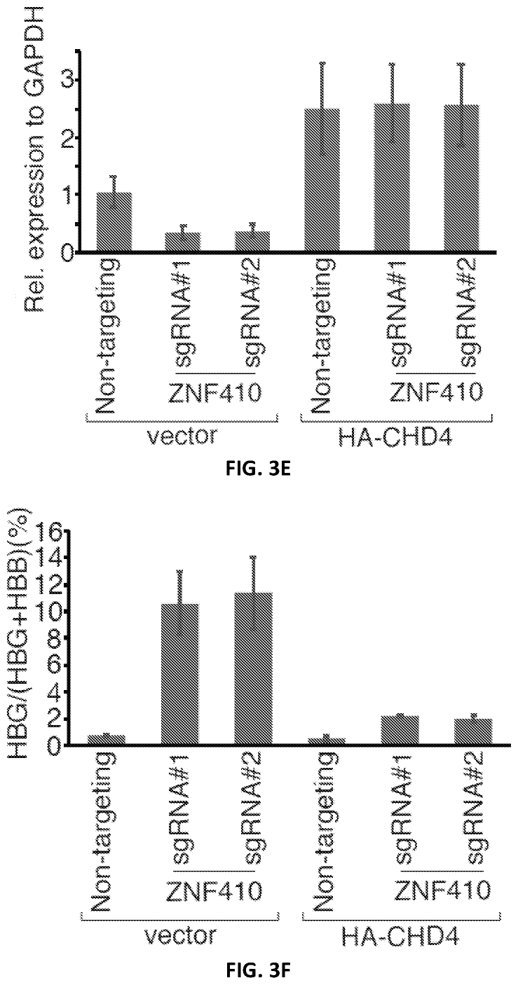

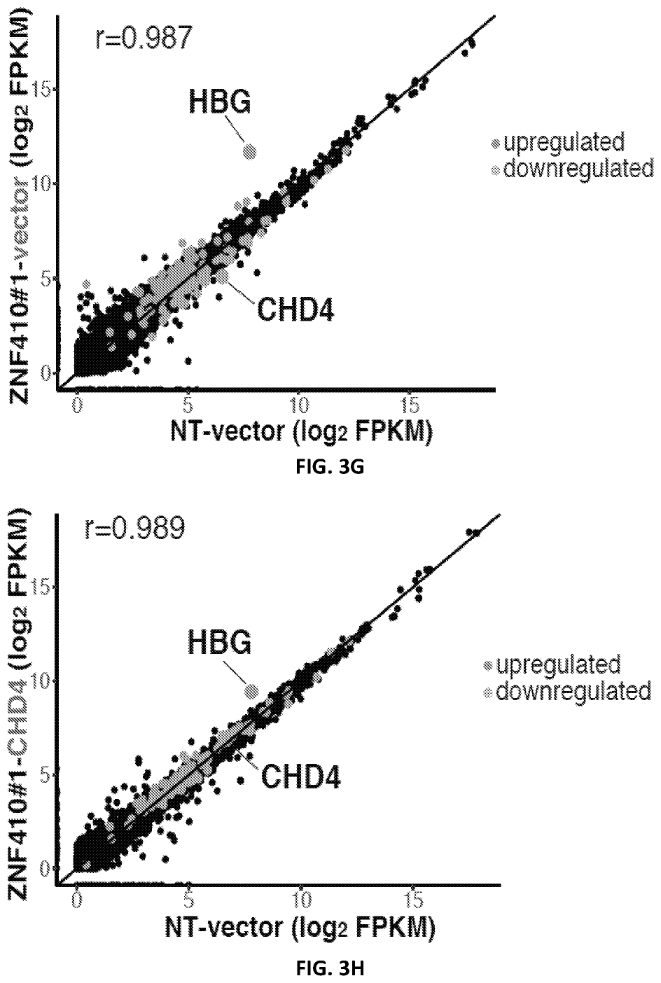

ZNF410 functions as a transcriptional activator (Benanti, et al. (2002) Mol. Cell Biol., 22:7385-7397). To understand how ZNF410 regulates the transcription of γ-globin genes, RNA-seq experiments were performed in ZNF410 depleted differentiated HUDEP-2 cells and primary human erythroblasts. Upon ZNF410 depletion in HUDEP2 cells, 70 genes were up- and 46 genes were down-regulated, respectively, with a threshold setting of 1.5-fold (p-value<0.05), and only counting genes that incurred changes with both ZNF410 sgRNAs in each of the biological replicates. In primary erythroid cultures, 83 genes were up-regulated and 126 genes were down-regulated, respectively upon ZNF410 depletion. This includes 30 up-regulated and 15 down-regulated genes in both cell types. Notably, γ-globin (HBG) mRNA levels stood out among the most strongly induced genes (FIGS. 3A-3B, Table 2).

TABLE 2
Differentially expressed transcripts from HUDEP-2 and erythroblasts by
RNA-seq. Z#1: ZNF410 sgRNA#1; Z#2: ZNF410 sgRNA#2; NT: Non-targeting.

| HUDEP-2 cells | | Primary CD34+ cells | |
|---|---|---|---|
| Top10 upregulated genes | log2(Z#2/NT) | Top10 upregulated genes | log2(Z#2/NT) |
| HBG1/2 | 4.28 | HSPB1 | 2.15 |
| AC104389.5 | 4.01 | IFI27 | 1.78 |
| RNU5B-1 | 3.60 | HBG1/2 | 1.75 |
| SNORD97 | 2.29 | SELENOW | 1.67 |
| CKB | 2.24 | RN7SL471P | 1.60 |
| PRG2 | 2.17 | PKN1 | 1.43 |
| H1F0 | 2.13 | AC104389.5 | 1.43 |
| AK1 | 1.83 | STUB1 | 1.40 |
| RNU5E-1 | 1.82 | PAFAH1B3 | 1.40 |
| CD63 | 1.75 | CDKN1A | 1.38 |
| | log2(Z#1/NT) | | log2(Z#1/NT) |
| HBG1/2 | 4.26 | HSPB1 | 2.01 |
| AC104389.5 | 3.95 | SELENOW | 1.55 |
| CKB | 2.44 | HBG1/2 | 1.47 |
| H1F0 | 2.41 | STUB1 | 1.46 |
| PRG2 | 2.09 | RPS19BP1 | 1.42 |
| AK1 | 1.99 | PAFAH1B3 | 1.35 |
| MIF | 1.92 | ISOC2 | 1.34 |
| CD63 | 1.90 | PKN1 | 1.32 |
| TMSB10 | 1.79 | COA4 | 1.30 |
| ATF5 | 1.65 | DYNLT1 | 1.27 |
| Top10 downregulated genes | log2(Z#2/NT) | Top10 downregulated genes | log2(Z#2/NT) |
| FOS | −1.88 | AC130456.3 | −2.03 |
| S100A10 | −1.59 | CBX6 | −2.01 |
| AC092490.1 | −1.50 | TMCC2 | −1.69 |
| SLC2A3P1 | −1.49 | AC092490.1 | −1.47 |
| SCARNA3 | −1.30 | ACSL6 | −1.47 |
| CHD4 | −1.23 | KDM7A | −1.32 |
| SCARNA18 | −1.20 | ZNF410 | −1.24 |
| RNU6-11P | −1.11 | SMOX | −1.24 |
| TUBA1A | −1.07 | PRDX5 | −1.09 |
| PLA2G6 | −1.01 | CHD4 | −1.08 |
| | log2(Z#1/NT) | | log2(Z#1/NT) |
| RF00019 | −2.43 | TMCC2 | −2.01 |
| S100A10 | −1.60 | ACSL6 | −1.67 |
| AC092490.1 | −1.45 | CBX6 | −1.56 |
| FOS | −1.32 | AC092490.1 | −1.50 |
| TMCC2 | −1.18 | ZNF410 | −1.49 |
| GLIPR2 | −1.14 | SMOX | −1.45 |
| TUBA1A | −1.06 | PRDX5 | −1.00 |
| CHD4 | −0.99 | CHD4 | −0.93 |
| PPP4R1 | −0.95 | SPC25 | −0.93 |
| SCARNA3 | −0.89 | GPCPD1 | −0.93 |

The CHD4 gene, which encodes a catalytic subunit of the NuRD complex, was among the most downregulated genes (FIGS. 3A-3B, Table 2). The NuRD complex contributes to the γ-globin repressive functions of BCL11A and LRF in erythroid cells. In addition to CHD4, the GATAD2A, HDAC2, MBD2 and MTA2 subunits of the NuRD complex are required for γ-globin repression (Sher, et al. (2019) Nat. Genet., 51:1149-1159). However, the mRNA levels of these subunits were not diminished in ZNF410 depleted cells, indicating that CHD4 is the only ZNF410-regulated NuRD subunit (FIGS. 3A-3B). These results were validated by RT-qPCR in HUDEP-2 cells, cultured primary erythroid cells, and ZNF410-depleted erythroid cells isolated from xenotransplanted NBSGW mice. Overall, the reduction in CHD4 transcript levels amounted to approximately 65% in HUDEP-2, primary cultures and xenotransplanted mice. That ZNF410 is limiting for CHD4 transcription is further supported by the strong correlation between ZNF410 and CHD4 transcript levels across 53 human tissues based on the Genotype-Tissue Expression database. Lastly, neither ZNF410 nor CHD4 have any developmental stage specificity as their mRNA levels are comparable between fetal and adult erythroblasts (Huang, et al. (2017) Genes Dev., 31:1704-1713).

In agreement with the mRNA analysis, CHD4 protein levels were significantly reduced upon ZNF410 depletion in HUDEP-2 and primary erythroblasts, while BCL11A, LRF, HDAC2 and MBD2 protein amounts remained unchanged (FIGS. 3C and 3D). Of note, although GATAD2A and MTA2 transcripts were unaltered, their protein levels were reduced upon ZNF410 depletion (FIGS. 3C-3D), which could also contribute to the induction of γ-globin. These subunits may be destabilized in the absence of CHD4 (Torrado, et al. (2017) FEBS J., 284:4216-4232). No other genes known to regulate γ-globin silencing were altered by ZNF410 depletion, indicating that CHD4 is the critical link between ZNF410 and γ-globin silencing.

CHD4 is the Sole Mediator of ZNF410 Function

The results implicate CHD4 as the key ZNF410-controlled regulator of γ-globin silencing. Therefore, it was examined whether expression of CHD4 in ZNF410 depleted cells restored γ-globin silencing. ZNF410-deficient HUDEP-2 cells were transduced with a lentiviral vector encoding CHD4 cDNA linked to an IRES element and GFP expression cassette, followed by FACS purification of GFP+ cells. The transduced cells expressed CHD4 mRNA at a level approximately 2.0-fold above normal (FIG. 3E). CHD4 expression almost completely restored the silencing of γ-globin without influencing the expression of other erythroid genes such as α-globin, β-globin, and GATA1 (FIG. 3F). To assess whether other transcriptional changes resulting from ZNF410 loss are also attributable to lower CHD4 levels, replicate RNA-seq experiments were performed in the CHD4-expressing ZNF410-deficient HUDEP-2 cells. Notably, 69 out of 70 upregulated genes and 44 out of 46 downregulated genes in the ZNF410 deficient cells were expressed at normal levels following CHD4 “rescue” (FIGS. 3G-3H; similar results were observed for ZNF410 #2). The expression of three genes (RNU2-2P, RNU4-2 and VSIR) was incompletely restored upon CHD4 re-expression. Without being bound by theory, this might be due to imperfect levels of CHD4 restoration or a drift in gene expression profiles following gene knockout/rescue experiments in cell pools. None of the three genes whose expression remained unrestored to normal levels are associated with ZNF410 ChIP-seq peaks, indicating that they are not direct ZNF410 targets. Together, these results indicate that CHD4 is the only functionally relevant ZNF410 target gene, and is responsible for the repression of γ-globin transcription. Another remarkable finding is that the γ-globin genes (HBG1/2) are among the most sensitive to CHD4 levels.

A Singular Enrichment of ZNF410 Binding Clusters at the CHD4 Gene

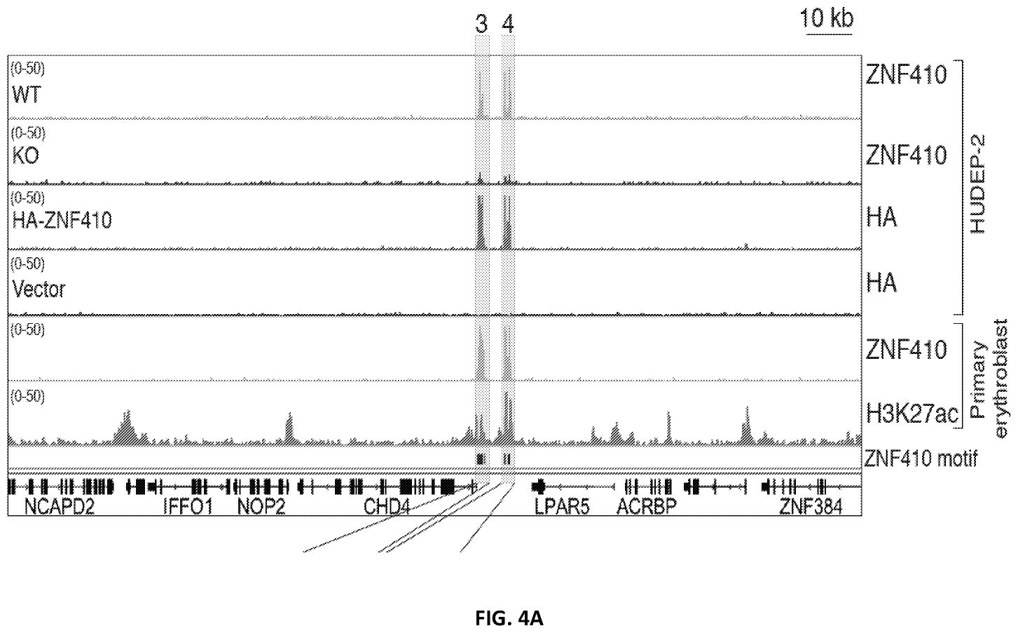

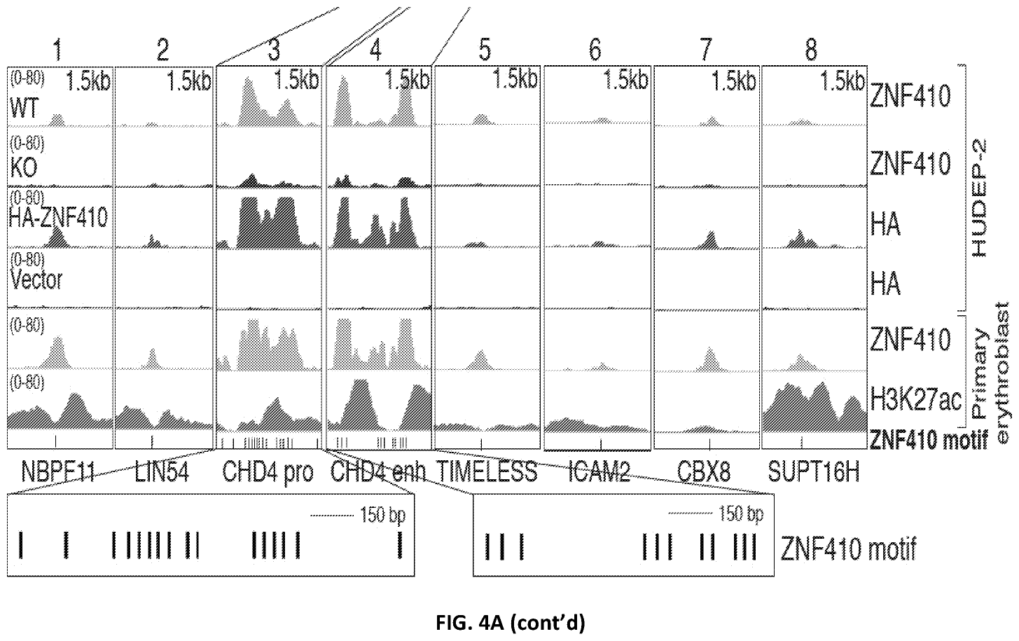

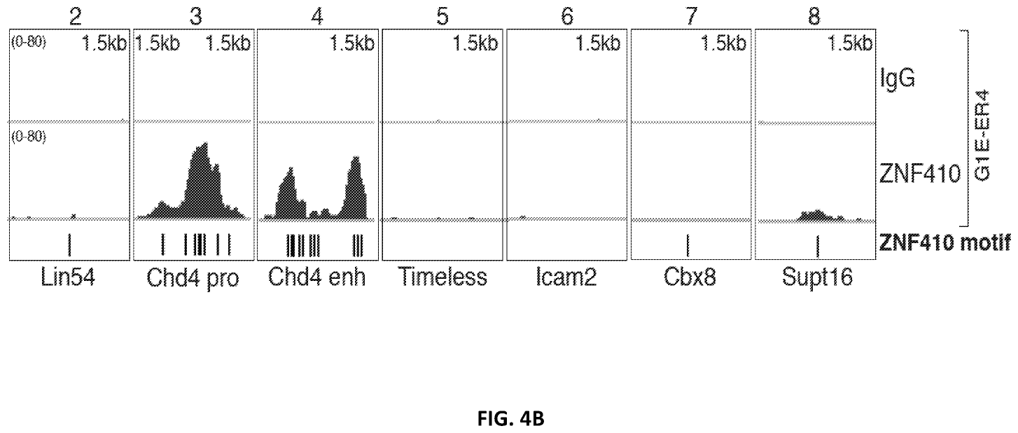

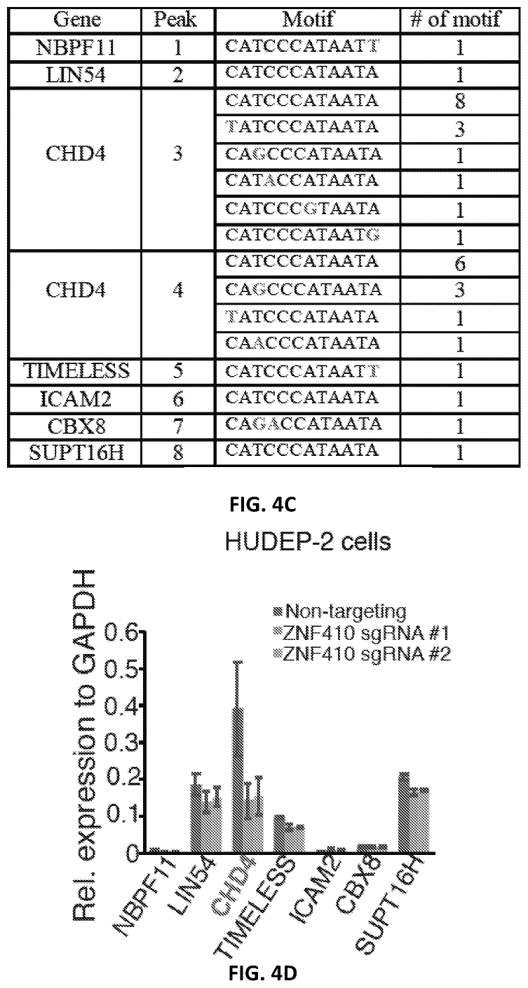

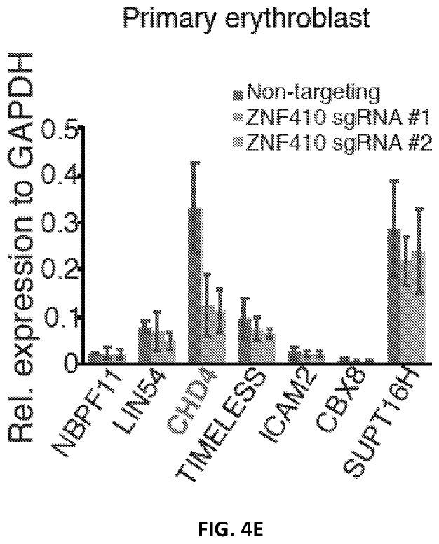

The RNA-seq study identified numerous genes that were up- or down-regulated after ZF410 depletion. To investigate which of these genes are direct ZF410 targets, anti-ZNF410 ChIP-seq was performed in HUDEP-2 and primary human erythroblasts with ZNF410-deficient HUDEP-2 cells as a control. Only 8 high-confidence peaks corresponding to 7 genes total were detected in both HUDEP-2 and primary human erythroid cells (FIG. 4A). To exclude the possibility that such unusually few called peaks are due to limitations in ZNF410 detection by ChIP, HA-tagged ZNF410 or empty vector in HUDEP-2 cells was overexpressed. Anti-HA ChIP-seq detected the same 8 ZNF410 peaks with comparable intensity profiles (FIG. 4A). No ZNF410 binding was detected at the β-globin locus, supporting a model in which ZNF410 regulates γ-globin transcription indirectly. 6 of 8 ZNF410 ChIP-seq peaks were of modest magnitude. Most strikingly, two very strong peaks were located at the promoter and enhancer of the CHD4 gene. These data indicate that ZNF410 directly regulates an unusually small number of genes and that suppression of ZNF410 may induce γ-globin transcription by downregulating the NuRD component CHD4.

HOMER motif analysis based on the 8 high-confidence binding sites from the ChIP-seq data generated the 12-nucleotide motif CATCCCATAATA (SEQ ID NO: 18) (FIG. 4C), which is almost identical to that found by in vitro SELEX experiments of human ZNF410 (Jolma, et al. (2013) Cell 152:327-339). Utilizing EMBOSS fuzznuc, 434 of such motif instances were found, and 3677 instances were found when combining all the motifs found under ZNF410 ChIP-seq peaks. Overall frequency of these motifs is very low compared to those of most transcription factor binding sites (Srivastava, et al. (2020) Biochim. Biophys. Acta Gene Regul. Mech., 1863:194443). However, since the vast majority of these motifs had no measurable ChIP-seq signal, additional features must account for the rare in vivo binding events. The two strongest ZNF410 ChIP-seq peaks were at promoter proximal (approximately-200 bp) and distal (approximately 6 kb) CHD4 regulatory regions, encompassing 15 and 11 motifs, respectively (each within a 1.5 kb window), while the remaining 6 modest peaks harbor only one motif (FIGS. 4A and 4C). Importantly, the two peaks at the CHD4 locus are the only regions in the entire genome with a high density of ZNF410 motifs, explaining the exquisite target specificity.

To explore additional criteria that might account for the selectivity of ZNF410 binding to chromatin, it was determined whether ZNF410 chromatin occupancy is associated with features of open chromatin. First, ChIP-seq profiles for H3K27ac, a histone mark associated with active chromatin, were generated in primary human erythroblasts and these data were complemented by mining chromatin accessibility (ATAC-seq) data from primary human erythroblasts (Ludwig, et al. (2019) Cell Reports 27:3228-3240). All 8 ZNF410 peaks, including the two strong peaks at the CHD4 promoter and enhancer, fell into accessible chromatin (based on ATAC-seq signal) that was also enriched in H3K27ac (FIG. 4A). In contrast, the vast majority of the unbound consensus motif instances elsewhere in the genome were in regions devoid of H3K27ac or ATAC-seq signal (Table 3). Only a very modest positive correlation between strengths of signal for ZNF410 binding with H3K27ac levels or ATAC-seq signal were found (Pearson's correlation coefficient of 0.33 and 0.24, respectively in primary erythroblasts). However, the categorical association of ZNF410 binding with open, active chromatin was almost complete, indicating that ZNF410 requires open chromatin and perhaps additional transcription factors, in addition to motif clustering, to enable its binding to chromatin.

TABLE 3

| | # of motif in | H3K27ac | ATAC signal | |
|---|---|---|---|---|
| Motif | human genome | only | only | Both |
| CATCCCATAATA | 434 | 11 | 13 | 15 |
| TATCCCATAATA | 561 | 17 | 9 | 3 |
| CAGCCCATAATA | 529 | 20 | 15 | 5 |
| CAACCCATAATA | 464 | 13 | 9 | 2 |
| CATCCCATAATT | 484 | 15 | 7 | 4 |
| CAGACCATAATA | 351 | 9 | 9 | 2 |
| CATACCATAATA | 422 | 11 | 6 | 2 |
| CATCCCGTAATA | 33 | 2 | 2 | 1 |
| CATCCCATAATG | 399 | 13 | 11 | 2 |

Analysis of H3K27ac ChIP-seq and ATAC signal from primary
human erythroid cells at ZNF410 motifs. Sequences from
top to bottom are SEQ ID NO: 18, SEQ ID NO: 61, SEQ ID
NO: 62, SEQ ID NO: 66, SEQ ID NO: 60, SEQ ID NO: 67,
SEQ ID NO: 63, SEQ ID NO: 64, and SEQ ID NO: 65.

The mere occupancy of a transcription factor at a gene does not necessarily lead to regulatory influence. The RNA-seq data in ZNF410-deficient cells was examined and validated for the expression of the seven genes bound by ZNF410. Importantly, among these genes, CHD4 was the only one with significantly reduced mRNA levels in ZNF410 depleted cells (FIGS. 4D-4E). Thus, ZNF410 directly and functionally regulates a single target gene, CHD4, in erythroid cells. Hence, the other gene expression changes that occur upon ZNF410 loss are likely due to diminished CHD4 levels. This model is supported by the restoration of transcriptome changes upon CHD4 expression in ZNF410 deficient cells (FIGS. 3G-3H).

ZNF410 Binding to Chromatin Occurs at Highly Conserved Motif Clusters

Highly conserved non-coding elements can function as enhancers and are associated with transcription factor binding sites (Pennacchio, et al. (2006) Nature 444:499-502). Conservation of the ZNF410 binding regions at the CHD4 locus was assessed using the phastCons scores deduced from sequence similarities across 100 vertebrate species (Siepel, et al. (2005) Genome Res., 15:1034-1050). Both ZNF410 binding site clusters display a high degree of conservation, comparable to that at the CHD4 exons. Moreover, the human ZNF410 protein sequence is 94% identical to mouse protein, and the DNA binding ZF domain is nearly 100% identical (FIG. 7D).

To examine whether ZNF410 binding selectivity for the CHD4 locus is conserved in mouse, ZNF410 ChIP-seq was performed in the erythroid cell line G1E-ER4 (Weiss, et al. (1997) Mol. Cell Biol., 17:1642-1651). As in human cells, the Chd4 proximal and distal regulatory regions were by far the most strongly ZNF410 occupied sites genome wide (FIG. 4B). Of the 6 human genes that exhibited modest ZNF410 ChIP-seq signals, no signal was detected at 4 orthologues in mice (Lin54, Timeless, Icam2, and Cbx8). A modest signal was detected at the mouse Supt16 gene but its expression was not altered by loss of ZNF410. Further analysis confirmed that among 1876 motif instances matching the most common motifs in the human genome, the Chd4 proximal and distal regulatory regions are also by far the most enriched locations in mouse genome (FIG. 4B). Taken together, these findings indicate that regulation of the CHD4 gene by ZNF410 is mediated through unique, evolutionarily conserved motif clusters.

Characterization of DNA Binding by ZNF410

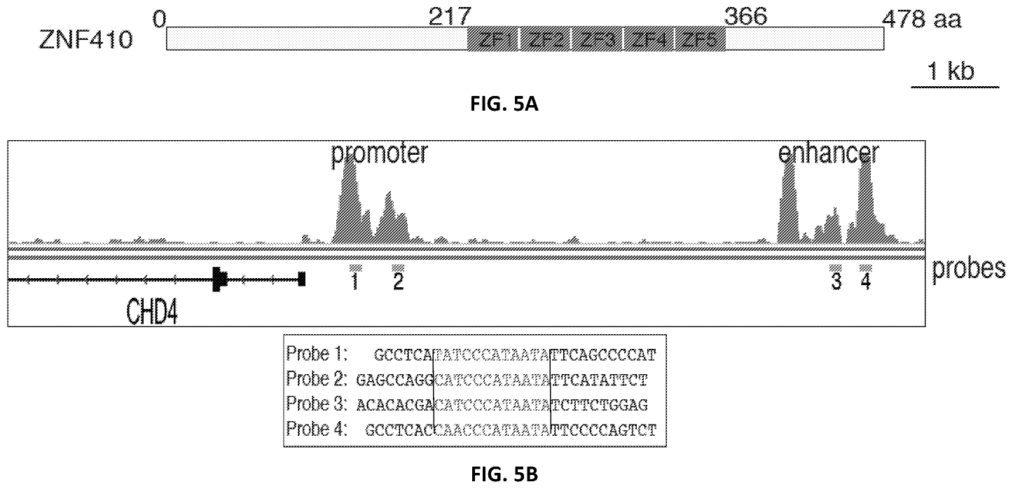

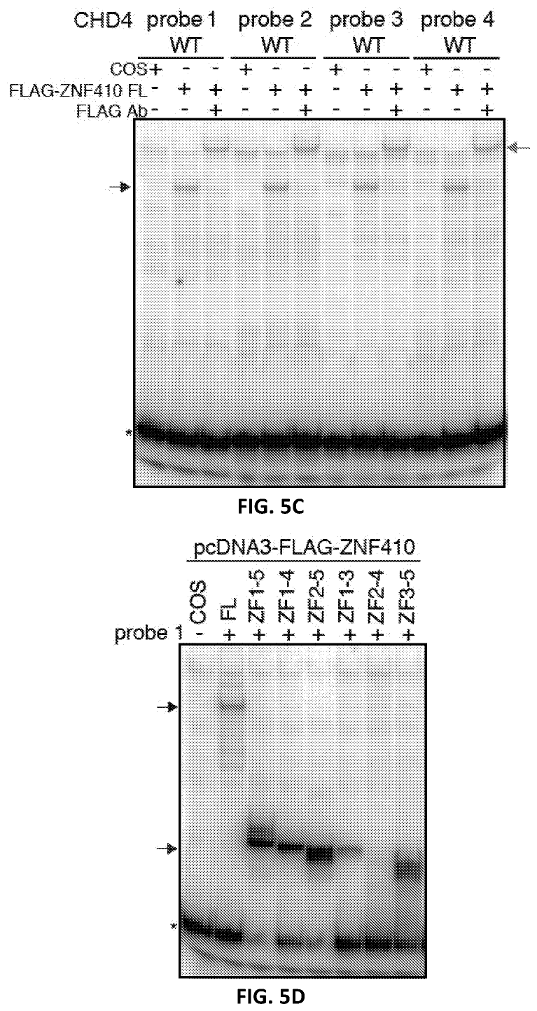

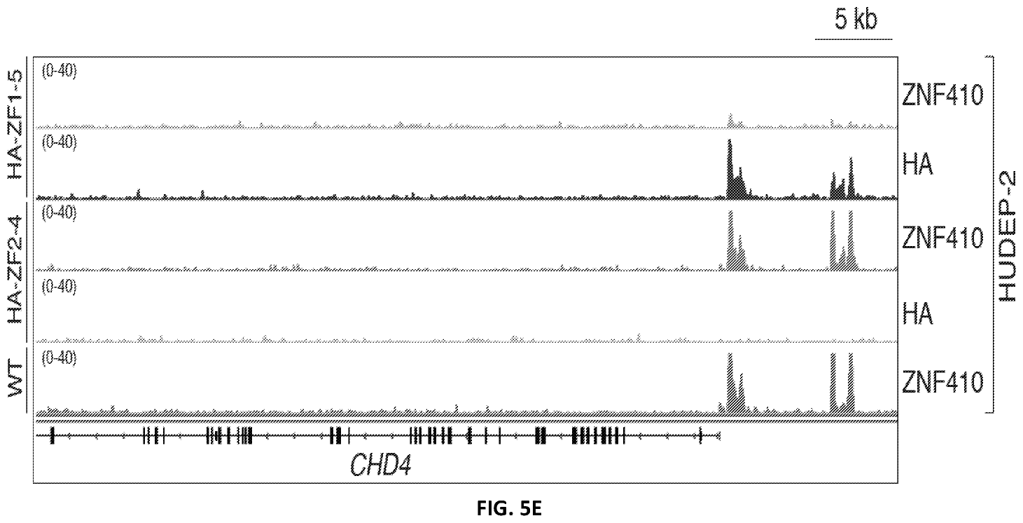

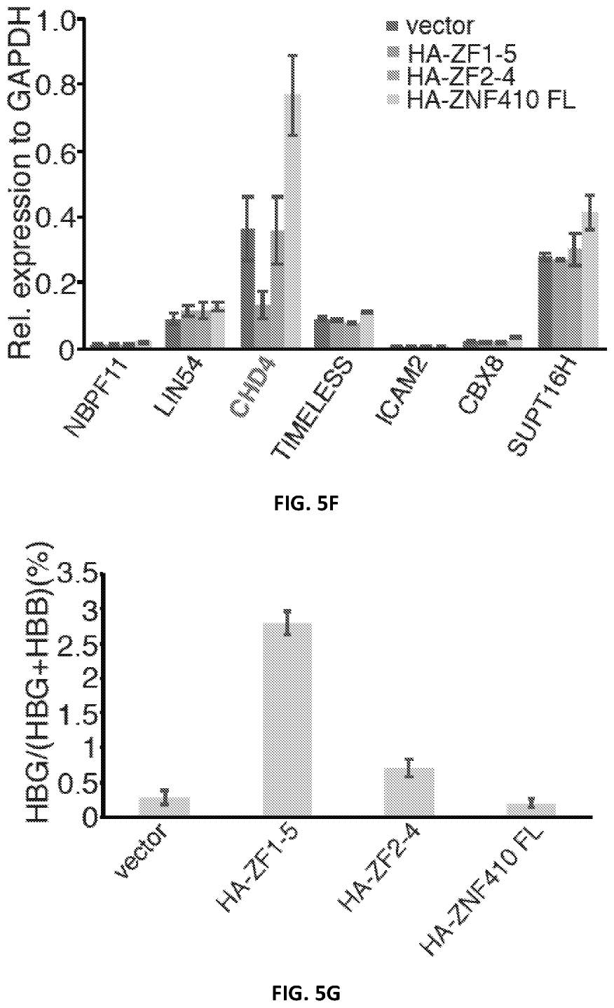

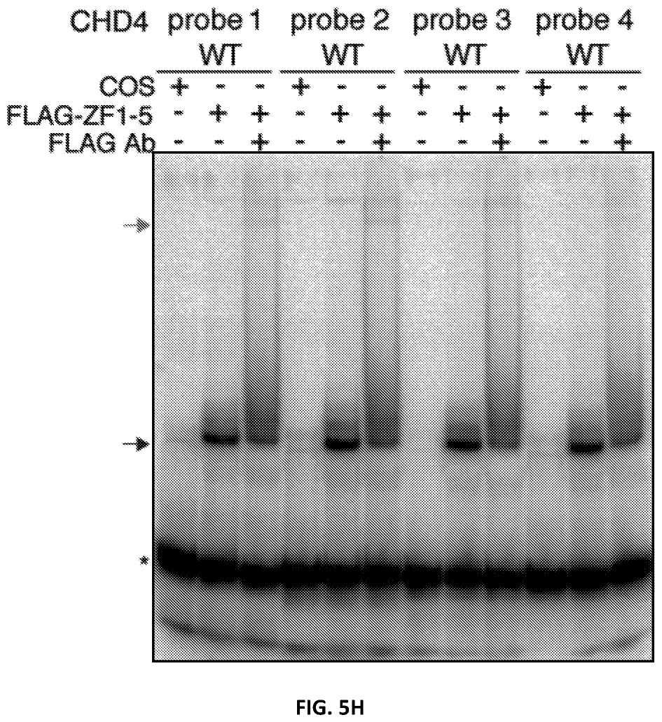

ZNF410 contains five tandem C2H2-type zinc fingers (ZFs) potentially involved in DNA binding (FIG. 5A). However, as is the case for many ZF transcription factors, not all ZFs necessarily make direct DNA contacts. Direct DNA binding by full length (FL) ZNF410 to sequences found at the CHD4 gene was assessed by electrophoretic mobility shift assays (EMSAs). Using nuclear extracts from COS cells overexpressing FLAG-tagged ZNF410 constructs and radiolabeled probes containing the relevant motifs (FIG. 5B), FL ZNF410 protein displayed comparable binding to each of the four DNA probes containing a single motif associated with ChIP-seq signal at the CHD4 promoter and enhancer (FIG. 5C). Addition of anti-FLAG antibody to the binding reaction led to a “supershift” (FIG. 5C), confirming binding specificity.

The domain spanning all five ZFs was sufficient for DNA binding, and like FL ZNF410, displayed similar binding intensity across the four probes (FIG. 5H). Again, addition of anti-FLAG antibody caused a “supershift”, validating the specific interactions between the ZF domain and the probes (FIG. 5H). The stronger signal generated by the ZF domain compared to that of full length ZNF410 (FIG. 5D) is likely due to the higher expression level of the former. To assess the contribution to DNA binding by each of the five ZFs, versions containing various ZF combinations were generated (FIG. 5D). In EMSA, the central 3 ZFs (2-4) were insufficient for DNA binding, however, if either ZF1 or ZF5 was present (ZF1-4 and ZF2-5, respectively), DNA binding was enabled (FIG. 5D). Additionally, DNA binding activity was observed, albeit reduced, by ZF1-3 and ZF3-5 (FIG. 5D). Thus, the central 3 ZFs (2-4) are insufficient for DNA binding, with a ZF at either end (ZF1 or ZF5) contributing to DNA contacts in this assay. Together, these data indicate that each of the five ZFs of ZNF410 is involved in DNA binding.

According to the EMSAs, the ZF1-5 domain displays strong DNA binding in vitro, while ZF2-4 does not (FIG. 5D). Overexpression of ZF1-5 but not ZF2-4 should compete with endogenous ZNF410 for chromatin binding, thus acting in a dominant-negative manner. To test this hypothesis, a construct containing HA-tagged ZF1-5 or ZF2-4 driven by the EF1α promoter was introduced via lentiviral infection into HUDEP-2 cells. As control, the expression of full-length HA-tagged ZNF410 was forced. Overexpression of ZF1-5 or ZF2-4 did not influence the endogenous ZNF410 expression. ChIP-seq experiments demonstrated that overexpressed ZF1-5 (roughly 20-fold compared to endogenous ZNF410 protein levels) bound to the CHD4 regulatory regions and to the other ZNF410 targets in a pattern very similar to endogenous ZNF410 (FIG. 5E). Moreover, full-length HA-ZNF410 when overexpressed to similar levels to HA-ZF1-5 also produced similar binding patterns (FIG. 4A). This indicates that the ZF domain is sufficient for ZNF410 chromatin occupancy, and that regions outside this domain contribute little if anything to chromatin binding. ZF2-4 displayed no chromatin occupancy at all sites examined, consistent with in vitro DNA binding properties, but with the caveat that it was expressed at lower levels (FIG. 5E). Accordingly, ZF1-5 markedly interfered with endogenous ZNF410 binding while ZF2-4 was inert (FIG. 5E). To assess the impact of interference with endogenous ZNF410 chromatin binding on CHD4 expression, RT-qPCR was performed and CHD4 mRNA levels were found to be reduced by approximately 70% compared to control (FIG. 5F), which is comparable to ZNF410 knockout (FIG. 3E). In contrast, overexpression of ZF2-4 did not influence CHD4 expression, however, overexpression of full length ZNF410 increased CHD4 expression (FIG. 5F). Thus, the ZF region is sufficient for chromatin occupancy but insufficient for CHD4 gene activation. In agreement with the results from ZNF410 depletion experiments, overexpression of ZF1-5 or ZF2-4 did not impact expression of the other 6 ZNF410 bound genes (FIG. 5F). Finally, the impact of ZNF410 expression on γ-globin levels was also measured. ZF1-5 expression triggered a significant increase in γ-globin mRNA levels (FIG. 5G), again comparable to that observed in ZNF410 depleted cells, while ZNF410 FL expression led to a slight decrease in the already low γ-globin mRNA levels (FIG. 5G). In sum, the ZF domain of ZNF410 is necessary and sufficient to bind to DNA in vitro and in vivo and does not seem to bear any transactivation function on its own.

Structural Basis of ZNF410 DNA Binding

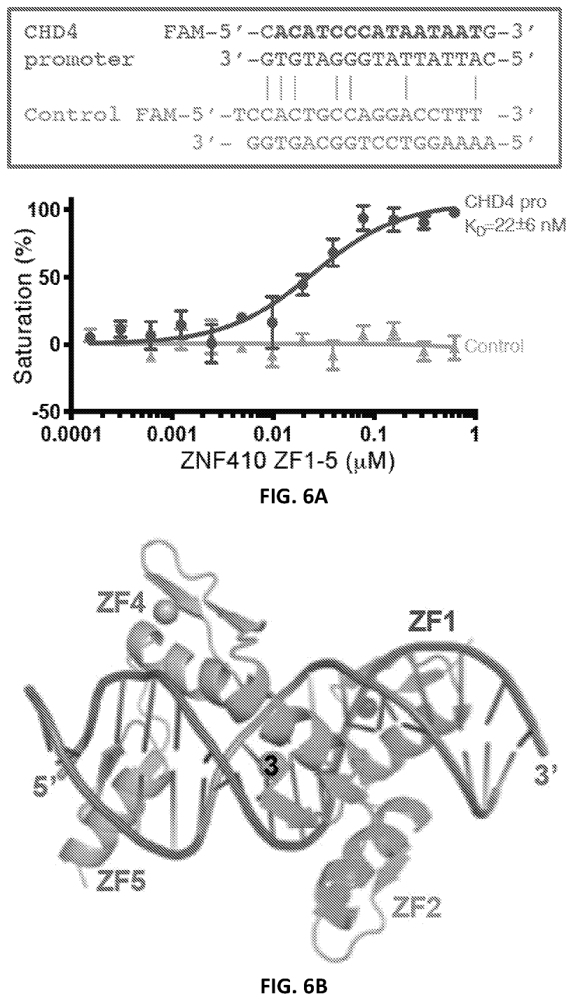

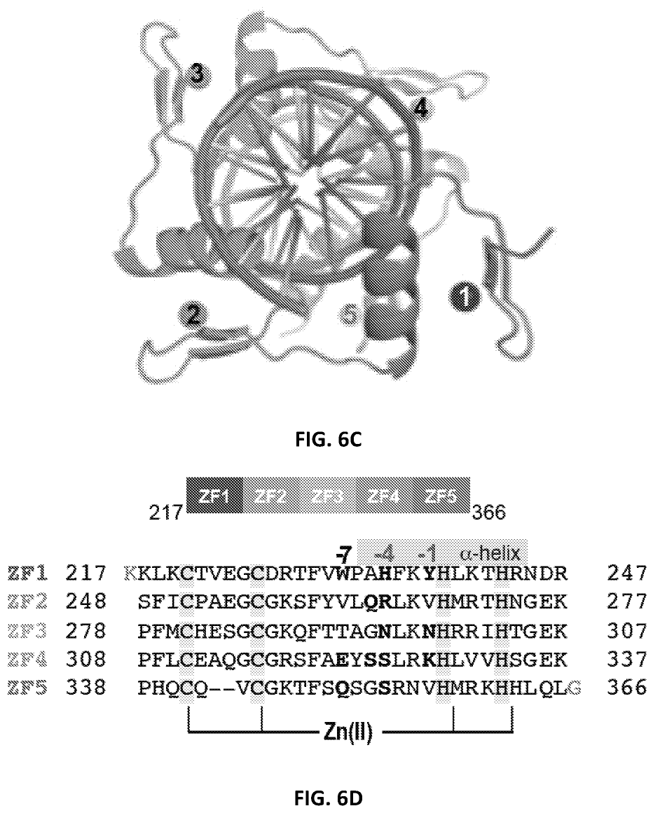

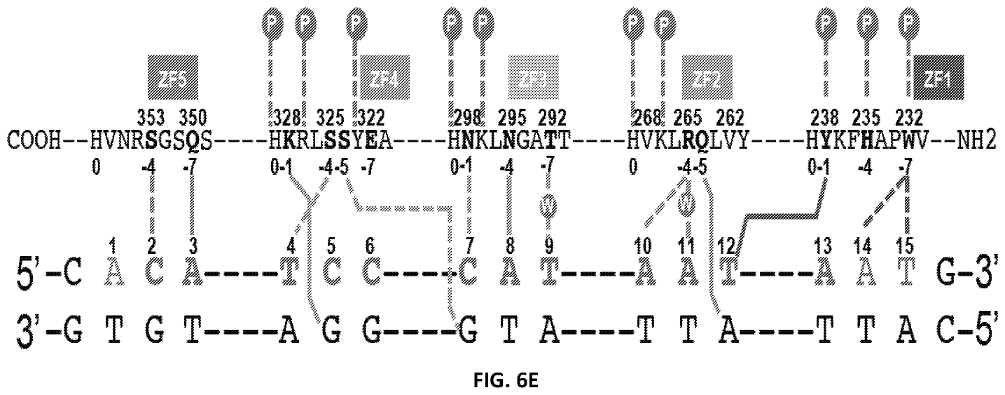

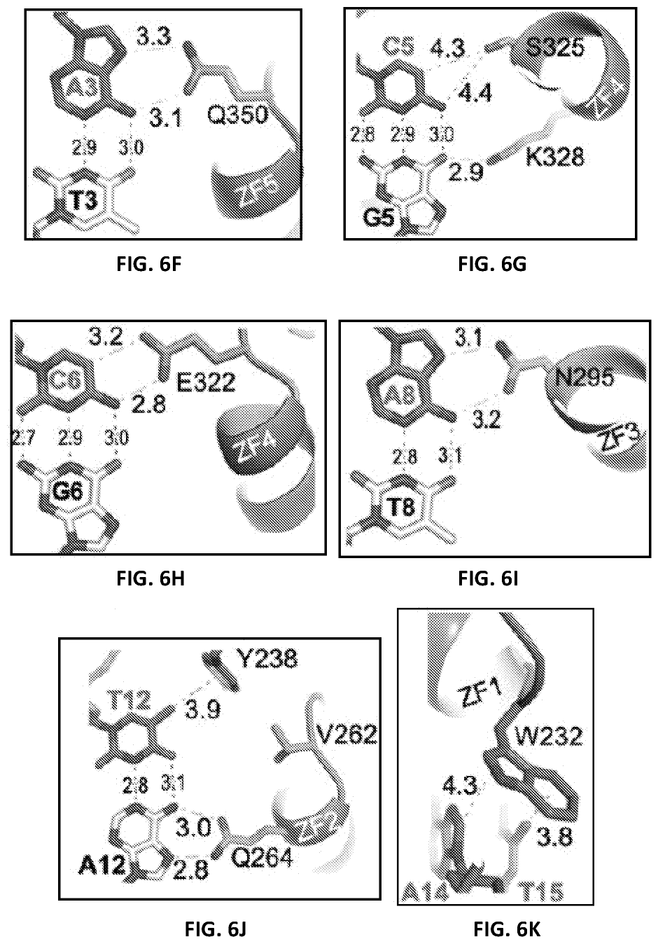

To further gain insight into the molecular basis of how the ZNF410 tandem ZF domain recognizes its targeting DNA sequence, crystallization of the ZNF410-DNA complex was performed. The binding affinity of the ZNF410 ZF domain (ZF1-5) with the consensus motif was quantified by fluorescence polarization using purified GST fusion protein (Patel, et al. (2016) Methods Enzymol., 573:387-401). The ZF domain displayed a dissociation constant (KD) of 22 nM for the oligo containing the consensus motif while there was no measurable binding to the negative control (that shares 7/17 bp with the consensus motif) under the same conditions (FIG. 6A). Using the same samples, the binding affinity between the ZF domain and the consensus oligonucleotide was confirmed to be between 10 and 20 nM by EMSA. Next, the crystal structure of the ZF domain in complex with the same 17-bp oligo (5′-3′: CACATCCCATAATAATG (SEQ ID NO: 19); 3′-5′: GTGTAGGGTATTATTAC (SEQ ID NO: 20)) containing the consensus motif was determined. The structure of the protein-DNA complex was solved by the single-wavelength anomalous diffraction (SAD) method (Hendrickson, et al. (1990) EMBO J., 9:1665-1672) at 2.75 Å resolution. As in conventional C2H2 ZF proteins (Wolfe, et al. (2000) Annu. Rev. Biophys. Biomol. Struct., 29:183-212), each of five fingers of ZNF410 comprises two strands and a helix, with two histidine residues in the helix together with one cysteine in each strand coordinating a zinc ion, forming a characteristic tetrahedral C2-Zn—H2 structural unit that confers rigidity to the fingers. When bound to DNA, ZF1-5 occupies the DNA major groove, with their α-helices toward DNA and the strands and the C2-Zn—H2 units facing outside (FIGS. 6B and C). Side chains from specific amino acids within the N-terminal portion of each helix and the preceding loop (i.e., the 7 residues prior to the first Zn-coordinating histidine; FIG. 6D) make major groove contacts with primarily three adjacent DNA base pairs (“triplet element”). The DNA oligo used for crystallization contains the 15-bp consensus sequence (numbered 1-15 from 5′ to 3′ of the top strand; FIG. 6E) recognized by the five fingers, plus one additional base pair on each end of the DNA duplex. The protein sequence runs in the opposite direction of the top strand, from carboxyl (COOH) to amino (NH₂) termini, resulting in ZF5 recognizing the 5′ triplet (bp position 1-3), and the ZF1 recognizing the 3′ triplet (bp position 13-15) (FIG. 6E).

Each zinc finger contributes to specific DNA interactions. The most dominant direct base-specific interactions observed are the Ade-Gln and Ade-Asn contacts via three fingers, e.g., Q350 of ZF5 interaction with A3 (FIG. 6F), N295 of ZF3 interaction with A8 (FIG. 6I), and Q264 of ZF2 interaction with A12 (FIG. 6J). In accordance with apposition of Gln/Asn with Ade as the most common mechanism for Ade recognition (Luscombe, et al. (2001) Nucleic Acids Res., 29:2860-2874), the side chain carboxamide moiety of glutamine and asparagine donates a H-bond to the 07 and accepts a H-bond from the N6 atoms of adenines, respectively, a pattern specific to Ade. ZF4 contacts two C:G base pairs at positions 5 and 6. K328 of ZF4 interacts with the O6 atom of G5 (FIG. 6G), while E322 of ZF4 forms a H-bond with the N4 atom and a C-H···O type H-bond (Horowitz, et al. (2012) J. Biol. Chem., 287:41576-41582) with the C5 atom of cytosine at position 6 (FIG. 6H). In addition, 5325 of ZF4 forms a van der Waals contact with cytosine at position 5 (FIG. 6G). ZF1 uses two aromatic residues (Tyr238 and Trp232) for interaction with the methyl group of thymine at position 12 and 15, respectively (FIGS. 6J and 6K). Among the base specific interactions, the C:G base pair at position 5 and the T:A base pair at position 12 have direct protein interactions with both bases (FIGS. 6G and 6J). In sum, the base specific interactions protect 10 base pairs (positions A3 to T12 in FIG. 6E), out of 12-base pair consensus sequence (FIG. 4C).

In addition to the direct base interactions, the first four fingers (ZF1-4) interact with DNA backbone phosphate groups, while ZF5 is devoid of such contact (FIG. 6E). Among the five ZFs, ZF5 has the least number of contacts with DNA, whereas ZF1 has only the van der Waals contacts with the bases, and two of them are outside of consensus (nucleotide positions 14 and 15 in FIG. 6E). This observation might explain that DNA binding in vitro was still enabled if either outside finger (ZF1 or ZF5), but not both, are removed (FIG. 5D). The structure also revealed that the fingers in the middle (ZF2-4) follow the one-finger-three base rule, each involving highly base specific interactions, whereas the fingers in the ends vary from 2-base (ZF5) to 4-base contacts (ZF1).

By leveraging an improved CRISPR-Cas9 screening platform, ZNF410, a pentadactyl zinc finger protein, was identified as a novel regulator of fetal hemoglobin expression. ZNF410 regulates γ-globin expression through selective activation of CHD4 transcription. CHD4 appears to be the only direct functional target of ZNF410 in erythroid cells. Two highly conserved clusters of ZNF410 binding sites at the CHD4 proximal and distal regulatory regions that appear to be unique in the human and mouse genomes account for selective accumulation of ZNF410 at the CHD4 locus. In the absence of ZNF410, CHD4 transcription is reduced but not entirely lost which explains the modest impact on global gene expression, and exposes the γ-globin genes as particularly sensitive to CHD4/NuRD levels. In vitro DNA binding assays and crystallography reveal the DNA binding modalities. This study thus illuminates a highly selective transcriptional pathway from ZNF410 to CHD4 to the γ-globin genes in erythroid cells.

Most transcription factors bind to thousands of genomic sites, of which a significant fraction trigger changes in gene transcription. ZNF410, however, directly activates just one gene in human erythroid cells. This is supported by the following observations. 1) ZNF410 chromatin binding as measured by ChIP-seq is only seen at a total of eight regions, with by far the strongest signals occurring in the form of two peak clusters near the CHD4 gene. Failure to detect more ChIP-seq peaks was not a consequence of overlooking potentially bound regions because of mappability issues, such as those presented by repetitive elements, since inclusion of reads that map to multiple locations did not reveal additional binding sites. 2) Clusters of ZNF410 motifs such as those at the CHD4 locus are not found elsewhere in the genome. 3) At the non-CHD4 ZNF410-bound sites, signals were not only much weaker, but showed little or no signal in murine cells. Hence, ZNF410 chromatin occupancy is conserved only at the CHD4 locus. 4) Among the few ZNF410 bound genes, CHD4 was the only one whose expression was reduced upon ZNF410 loss or upon expression of dominant interfering ZNF410 constructs. 5) Forced expression of CHD4 almost completely restored γ-globin silencing and transcriptome in ZNF410 deficient cells. This also indicates that indirect, motif-independent binding to chromatin, which might escape detection by ChIP, would not have significant regulatory influence.

When interrogating data sets from 53 tissues, the ZNF410 and CHD4 mRNA levels are highly correlated, indicating that ZNF410 may be generally limiting for CHD4 expression across tissues and cell lines. Notably, in luminal breast cancer cell lines, ZNF410 and CHD4 are the top co-essential genes, implying that they function in the same pathway (Depmap; depmap.org). Loss of ZNF410 does not completely abrogate CHD4 gene transcription. Consequently, the requirement of ZNF410 for CHD4 transcription is not absolute, implicating the possibility of the involvement of other factors in the regulation of the CHD4 gene.

There are no other transcriptional activators with single target genes, but there are cases of transcription factors with only very few target genes. For example, ZFP64 is an 11-zinc finger protein which binds most strongly to clusters of elements near the MLL gene, reminiscent of the ZNF410 motif clusters at the CHD4 locus (Lu, et al. (2018) Cancer Cell 34:970-981). Yet ZFP64 displays thousands of additional high confidence ChIP-seq peaks even though it regulates only a small fraction of associated genes. The KRAB-ZFP protein Zfp568 is a transcriptional repressor that seems to only silence the expression of the Igf2 gene in embryonic and trophoblast stem cells, even though it occupies dozens of additional sites in the genome (Yang, et al. (2017) Science 356:757-759). Remarkably, deletion of the Igf2 gene in mice rescues the detrimental effects on gastrulation incurred upon Zfp568 loss, but embryonic lethality persists, implying the presence of additional Zfp568 repressed genes. Extraordinarily high gene selectivity has also been reported for transcriptional co-factors. For example, TRIM33, a cofactor for the myeloid transcription factor PU.1, has been shown to occupy only 31 genomic sites in murine B cell leukemia, and appears to preferentially associate with enhancers containing a high density of PU.1 binding sites (Wang, et al. (2015) Elife 4:e06377). Transcription factors are normally employed at numerous genes, and spatio-temporal specificity is accomplished through combinatorial action with other transcription factors. However, the number of target genes for transcription factors and co-factors varies by three orders of magnitude (ENCODE Transcription Factor Targets). ZNF410 seems to have evolved to require motif clusters such as those found at the CHD4 locus to achieve such high levels of target gene specificity.

The high selectivity of ZNF410 chromatin occupancy can be accounted for by several factors. 1) The human genome contains 434 perfect ZNF410 motif instances and 3677 similar ones if adding up all the motifs that are found under ZNF410 peaks, which is a much smaller number than that for the great majority of transcription factors (Srivastava, et al. (2020) Biochim. Biophys. Acta Gene Regul. Mech., 1863:194443). Thus, motif scarcity is likely one determinant of target selectivity but obviously insufficient as the sole explanation. 2) ZNF410 binding site clusters are uniquely found at the CHD4 gene. If ZNF410 requires a cooperative mechanism for chromatin binding, this may explain lack of binding to the majority of single motifs. 3) The weak ZNF410 binding that is found at 6 sites containing a single motif is accompanied by the presence of active histone marks and signatures of open chromatin. It is possible that when exposed, single motifs might allow access to ZNF410 even if it is functionally inconsequential. Indeed, ZNF410 depletion or dominant interfering ZNF410 version elicited no transcriptional changes of the 6 ZNF410-bound genes with a single motif.

The ZF domain of ZNF410 is necessary and sufficient for DNA binding in vitro and in vivo. Crystallographic analysis of the ZF domain bound to DNA revealed a binding mode in which ZF1-ZF5 are contacting the consensus sequence in a 3′ to 5′ orientation with all five ZFs contacting DNA. EMSA experiments indicate, however, that four ZFs (either ZF1-4 or ZF2-5) are needed for efficient binding. Fluorescence polarization experiments measured the ZF domain-DNA interaction KD at 22 nM. Yet, this high affinity interaction appears insufficient to enable chromatin occupancy at virtually all single elements in the genome. Hence, the clustering of motifs may be required to convey efficient and high level chromatin binding. Since the ZF domain displays no activation activity on its own and therefore might not interact with co-activator complexes, binding cooperativity might derive from the inherently synergistic effects of DNA binding domains when displacing histone-DNA interactions in nucleosomes (Adams, et al. (1995) Mol. Cell Biol., 15:1405-1421; Oliviero, et al. (1991) Proc. Natl. Acad. Sci., 88:224-228; Polach, et al. (1996) J. Mol. Biol., 258:800-812).

When overexpressed, the ZF domain acted in a dominant interfering manner by displacing endogenous ZNF410 from the CHD4 locus. The resulting reduction in CHD4 transcription was ˜65%-70%, comparable to that observed upon ZNF410 knockout. The expression of 6 other ZNF410 bound genes were unaffected, again illustrating ZNF410 specificity. Without being bound by theory, one implication of this finding is that the transactivation function of ZNF410 resides outside the ZF domain, and that, by inference, the ZF domain may not be involved in co-activator recruitment. This contrasts with other zinc finger transcription factors, such as GATA1 where the ZF region can be multifunctional and not only bind DNA but also critical co-regulators (Campbell, et al. (2013) Blood 121:5218-5227). Finally, according to the ChIP-seq experiments, the ZF domain binding profiles are very similar to full length ZNF410, indicating that the ZNF410 chromatin binding specificity and affinity is determined solely by the ZF domain, and that other domains and associated cofactors contribute little if at all to ZNF410 binding.

Sequence variants at binding sites for the γ-globin repressors BCL11A and LRF (ZBTB7A) are linked to persistence of γ-globin expression into adulthood (Liu, et al. (2018) Cell 173:430-442; Martyn, et al. (2018) Nat. Genet., 50:498-503). Given the large number of ZNF410 elements at the CHD4 locus, multiple elements would need to be lost in order to significantly affect CHD4 transcription. It is thus possible that motif clustering at the CHD4 locus provides robustness for the maintenance of CHD4 expression.

Complete CHD4 loss severely compromises hematopoiesis and erythroid cell growth (Sher, et al. (2019) Nat. Genet., 51:1149-1159; Xu, et al. (2013) Proc. Natl. Acad. Sci., 110:6518-6523; Yoshida, et al. (2008) Genes Dev., 22:1174-1189). However, depletion of ZNF410 is well tolerated in erythroid cells and other hematopoietic lineages, which is likely due to the fact that CHD4 is not completely extinguished. This partial CHD4 reduction was sufficient to robustly de-repress the γ-globin genes (Amaya, et al. (2013) Blood 121:3493-3501). Notably, given the very limited global transcriptional changes upon ZNF410 depletion, this indicates that the γ-globin genes are especially sensitive to CHD4/NuRD levels.

In sum, ZNF410 was identified as a highly specific regulator of CHD4 expression and γ-globin silencing. This high transcriptional selectivity can be exploited and ZNF410 may be targeted to raise fetal hemoglobin expression for the treatment of hemoglobinopathies.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method of increasing the level of human fetal hemoglobin and/or γ-globin in a cell or subject, the method comprising administering at least one zinc finger protein 410 (ZNF410) inhibitor to the cell or subject.

2. The method of claim 1, wherein the subject has a β-chain hemoglobinopathy.

3. The method of claim 1, wherein the subject has thalassemia.

4. The method of claim 1, wherein the subject has sickle cell disease.

5. The method of claim 1, wherein the ZNF410 inhibitor is an inhibitory nucleic acid molecule.

6. The method of claim 5, wherein said inhibitory nucleic acid molecule is an siRNA, shRNA, or an antisense molecule.

7. The method of claim 1, wherein the ZNF410 inhibitor is a fragment of ZNF410 comprising at least four zinc finger domains or a nucleic acid molecule encoding the fragment.

8. The method of claim 1, wherein the ZNF410 inhibitor is a DNA binding domain inhibitor.

9. The method of claim 1, wherein the cell is an erythroid cell, progenitor cell, or stem cell.

10. The method of claim 1, further comprising administering at least one fetal hemoglobin inducer to the cell or subject.

11. The method of claim 10, wherein said fetal hemoglobin inducer is pomalidomide.

12. A method of treating a hemoglobinopathy in a subject in need thereof, the method comprising administering a composition comprising at least one zinc finger protein 410 (ZNF410) and a pharmaceutically acceptable carrier to the subject.

13. The method of claim 12, wherein the subject has a β-chain hemoglobinopathy.

14. The method of claim 12, wherein the subject has thalassemia.

15. The method of claim 12, wherein the subject has sickle cell anemia.

16. The method of claim 12, wherein the ZNF410 inhibitor is an inhibitory nucleic acid molecule.

17. The method of claim 16, wherein said inhibitory nucleic acid molecule is an siRNA, shRNA, or an antisense molecule.

18. The method of claim 12, wherein the ZNF410 inhibitor is a fragment of ZNF410 comprising at least four zinc finger domains or a nucleic acid molecule encoding the fragment.

19. The method of claim 12, wherein the ZNF410 inhibitor is a DNA binding domain inhibitor.

20. The method of claim 12, further comprising administering at least one fetal hemoglobin inducer to the subject.

21. The method of claim 20, wherein said fetal hemoglobin inducer is pomalidomide.

22. The method of claim 12, wherein the ZNF410 inhibitor is contained within a cell administered to the subject.


SEQUENCE LISTING (78 sequences)

SEQ ID NO: 1 - protein, 478 aa, Homo sapiens
MLSDELESKPELLVQFVQNTSIPLGQGLVESEAKDITCLSLLPVTEASECSRLMLPDDTTNHSNSSKEVPSSAVLRSLRVNVGPDGEETRAQTVQKSPEFLSTSESSSLLQDLQPSDSTSFILLNLTRAGLGSSAEHLVFVQDEAEDSGNDFLSSESTDSSIPWFLRVQELAHDSLIAATRAQLAKNAKTSSNGENVHLGSGDGQSKDSGPLPQVEKKLKCTVEGCDRTFVWPAHFKYHLKTHRNDRSFICPAEGCGKSFYVLQRLKVHMRTHNGEKPFMCHESGCGKQFTTAGNLKNHRRIHTGEKPFLCEAQGCGRSFAEYSSLRKHLVVHSGEKPHQCQVCGKTFSQSGSRNVHMRKHHLQLGAAGSQEQEQTAEPLMGSSLLEEASVPSKNLVSMNSQPSLGGESLNLPNTNSILGVDDEVLAEGSPRSLSSVPDVTHHLVTMQSGRQSYEVSVLTAVNPQELLNQGDLTERRT

SEQ ID NO: 2 - DNA, 1437 bp, Homo sapiens
atgttatcagatgagttagaatccaaaccagagctcctggtacagtttgttcagaatacgtccatcccattgggacaggggcttgtagaatcagaagctaaagatattacttgcttgtccctccttcccgtgactgaagcctcagaatgcagtcggctaatgttaccagatgatactacaaatcattctaactcctccaaggaggtcccttcctcagctgttttgagaagccttcgggtgaatgtgggtccagacggagaggagacgagagctcagactgtacagaaatccccggagtttttgtccacttcagagtcttctagcttgttgcaagatctacagccaagtgatagcacttcttttattcttcttaacctaacaagagcaggtctgggctcttcagctgagcacttagtgtttgtacaggatgaggcagaagattcagggaatgatttcctctccagtgagagcacagacagtagcattccatggttcctccgggttcaggagttggcccatgacagtttgattgctgctactcgtgcacaactggcaaagaatgcaaaaaccagcagcaatggagaaaatgtccaccttggttctggtgatgggcagtcaaaagattctgggccccttcctcaagtggaaaagaagctcaagtgtacagttgaaggttgtgaccggacatttgtatggccagctcactttaaataccacctcaagactcatcgaaatgaccgctccttcatctgtcctgcagaaggttgtgggaaaagcttctatgtgctgcagaggctgaaggtgcacatgaggacccacaatggagagaagccctttatgtgccatgagtctggctgtggtaagcagtttactacagctggaaacctgaagaaccaccggcgcatccacacaggagagaaacctttcctttgtgaagcccaaggatgtggccgttcctttgctgagtattctagcctccgaaaacatctggtggttcactcaggagagaagcctcatcagtgccaagtctgtgggaagaccttctctcagagtggaagcaggaatgtgcatatgagaaagcatcacctgcagctgggagcagctgggagtcaagagcaggagcaaactgctgagccactaatgggcagtagtttgcttgaagaggcttcagtacccagtaaaaacctggtgtctatgaattcccagcccagccttggtggagagtccttgaacctaccaaataccaattctatcctgggagttgatgatgaggtgcttgctgaaggatccccacgttccctgtcttcagtgcctgatgtgacacatcacctggtgaccatgcagtcagggaggcaatcatatgaagtttctgtcttaactgcagtaaatccacaagagttactaaaccaaggagatttaactgaaagacggacatga

SEQ ID NO: 3 - RNA, 80 nt, Artificial Sequence
  gRNA scaffold
guuuuagagcuagaaauagcaaguuaaaauaaggcuaguccguuaucaacuugaaaaaguggcaccgagucggugcuuuu

SEQ ID NO: 4 - DNA, 20 bp, Artificial Sequence
  ZNF410 sgRNA#1
gaaccaccagatgttttcgg

SEQ ID NO: 5 - DNA, 20 bp, Artificial Sequence
  ZNF410 sgRNA#2
ctcatcagtgccaagtctgt

SEQ ID NO: 6 - DNA, 20 bp, Artificial Sequence
  BCL11A+58 sgRNA
ctaacagttgcttttatcac

SEQ ID NO: 7 - DNA, 20 bp, Artificial Sequence
  Non-targeting sgRNA
gaccggaacgatctcgcgta

SEQ ID NO: 8 - DNA, 29 bp, Artificial Sequence
  ZNF410 sgRNA#1 Primer
gcctcatatcccataatattcagccccat

SEQ ID NO: 9 - DNA, 30 bp, Artificial Sequence
  ZNF410 sgRNA#1 Primer
gagccaggcatcccataatattcatattct

SEQ ID NO: 10 - DNA, 30 bp, Artificial Sequence
  ZNF410 sgRNA#2 Primer
acacacgacatcccataatatcttctggag

SEQ ID NO: 11 - DNA, 30 bp, Artificial Sequence
  ZNF410 sgRNA#2 Primer
gcctcaccaacccataatattccccagtct

SEQ ID NO: 12 - DNA, 21 bp, Artificial Sequence
  CHD4 promoter Primer
gcagaccttttgcaactaacc

SEQ ID NO: 13 - DNA, 20 bp, Artificial Sequence
  CHD4 promoter Primer
ggggtgcttattatgggatg

SEQ ID NO: 14 - DNA, 20 bp, Artificial Sequence
  CHD4 enhancer Primer
agcagccatcccataatagc

SEQ ID NO: 15 - DNA, 20 bp, Artificial Sequence
  CHD4 enhancer Primer
ctccatttcctctccagctc

SEQ ID NO: 16 - DNA, 21 bp, Artificial Sequence
  HBG2 Primer
tcacacacacacaaacacacg

SEQ ID NO: 17 - DNA, 20 bp, Artificial Sequence
  HBG2 Primer
agatgggggcaaagtatgtc

SEQ ID NO: 18 - DNA, 12 bp, Artificial Sequence
  ZNF410 binding motif
catcccataata

SEQ ID NO: 19 - DNA, 17 bp, Artificial Sequence
  ZNF410 binding motif
cacatcccataataatg

SEQ ID NO: 20 - DNA, 17 bp, Artificial Sequence
  ZNF410 binding motif
cattattatgggatgtg

SEQ ID NO: 21 - DNA, 18 bp, Artificial Sequence
  Control sequence
tccactgccaggaccttt

SEQ ID NO: 22 - DNA, 18 bp, Artificial Sequence
  Control sequence
aaaaggtcctggcagtgg

SEQ ID NO: 23 - DNA, 21 bp, Artificial Sequence
  Gamma_globin_F primer
tggcaagaaggtgctgacttc

SEQ ID NO: 24 - DNA, 20 bp, Artificial Sequence
  Gamma_globin_R primer
gcaaaggtgcccttgagatc

SEQ ID NO: 25 - DNA, 20 bp, Artificial Sequence
  Beta_globin_F primer
tgggcaaccctaaggtgaag

SEQ ID NO: 26 - DNA, 21 bp, Artificial Sequence
  Beta_globin_R primer
gtgagccaggccatcactaaa

SEQ ID NO: 27 - DNA, 21 bp, Artificial Sequence
  Alpha_globin_F primer
aagacctacttcccgcacttc

SEQ ID NO: 28 - DNA, 18 bp, Artificial Sequence
  Alpha_globin_R primer
gttgggcatgtcgtccac

SEQ ID NO: 29 - DNA, 20 bp, Artificial Sequence
  Gamma_globin_PT_F primer
tttgtggcaccttctgactg

SEQ ID NO: 30 - DNA, 18 bp, Artificial Sequence
  Gamma_globin_PT_R primer
gccaaagctgtcaaagaa

SEQ ID NO: 31 - DNA, 24 bp, Artificial Sequence
  MBD2_F primer
aagtgctggcaagagcgatgtcta

SEQ ID NO: 32 - DNA, 24 bp, Artificial Sequence
  MBD2_R primer
tttcccaggtaccttgccaactga

SEQ ID NO: 33 - DNA, 19 bp, Artificial Sequence
  GAPDH_F primer
agccacatcgctcagacac

SEQ ID NO: 34 - DNA, 19 bp, Artificial Sequence
  GAPDH_R primer
gcccaatacgaccaaatcc

SEQ ID NO: 35 - DNA, 20 bp, Artificial Sequence
  CHD4_F primer
gctgcaaccatccatacctc

SEQ ID NO: 36 - DNA, 20 bp, Artificial Sequence
  CHD4_R primer
accatcgatgcgttcgtatt

SEQ ID NO: 37 - DNA, 22 bp, Artificial Sequence
  GATA1_F primer
ctgtccccaatagtgcttatgg

SEQ ID NO: 38 - DNA, 22 bp, Artificial Sequence
  GATA1_R primer
gaataggctgctgaattgaggg

SEQ ID NO: 39 - DNA, 19 bp, Artificial Sequence
  MTA2_F primer
ttcccacctacactaagcc

SEQ ID NO: 40 - DNA, 19 bp, Artificial Sequence
  MTA2_R primer
aggcccttctgaaatccag

SEQ ID NO: 41 - DNA, 22 bp, Artificial Sequence
  BCL11A_F primer
acaaacggaaacaatgcaatgg

SEQ ID NO: 42 - DNA, 22 bp, Artificial Sequence
  BCL11A_R primer
tttcatctcgattggtgaaggg

SEQ ID NO: 43 - DNA, 22 bp, Artificial Sequence
  HDAC2_F primer
catggtgatggtgttgaagaag

SEQ ID NO: 44 - DNA, 24 bp, Artificial Sequence
  HDAC2_R primer
tcattggaaaattgacagcatagt

SEQ ID NO: 45 - DNA, 22 bp, Artificial Sequence
  GATAD2A_F primer
acgagttcatctacctggtcgg

SEQ ID NO: 46 - DNA, 22 bp, Artificial Sequence
  GATAD2A_R primer
acgtgaagtccgtcttgcactg

SEQ ID NO: 47 - DNA, 22 bp, Artificial Sequence
  NBPF11_F primer
gcatggctgttgacataggcag

SEQ ID NO: 48 - DNA, 23 bp, Artificial Sequence
  NBPF11_R primer
ctatccagtgagtcctgcaagac

SEQ ID NO: 49 - DNA, 23 bp, Artificial Sequence
  LIN54_F primer
gtccgacttgttactgccacatc

SEQ ID NO: 50 - DNA, 22 bp, Artificial Sequence
  LIN54_R primer
ttggaactgacatccgcactgg

SEQ ID NO: 51 - DNA, 22 bp, Artificial Sequence
  TIMELESS_F primer
aagtggtccaggtgtcggagaa

SEQ ID NO: 52 - DNA, 22 bp, Artificial Sequence
  TIMELESS_R primer
gtgggcactattctgctggtag

SEQ ID NO: 53 - DNA, 22 bp, Artificial Sequence
  ICAM2_F primer
atgacacggtcctccaatgcca

SEQ ID NO: 54 - DNA, 23 bp, Artificial Sequence
  ICAM2_R primer
gcactcaatggtgaaggacttgc

SEQ ID NO: 55 - DNA, 22 bp, Artificial Sequence
  CBX8_F primer
aacatcctggatgctcgcttgc

SEQ ID NO: 56 - DNA, 23 bp, Artificial Sequence
  CBX8_R primer
tttgaggaggaaggttttgggct

SEQ ID NO: 57 - DNA, 23 bp, Artificial Sequence
  SUPT16H_F primer
cattggtgacacagtgcttgtgg

SEQ ID NO: 58 - DNA, 22 bp, Artificial Sequence
  SUPT16H_R primer
ccaaaaggtcctctgcctcatc

SEQ ID NO: 59 - protein, 431 aa, Homo sapiens
MLSDELESKPELLVQFVQNTSIPLGQGLVESEAKDITCLSLLPVTEASECSRLMLPDDTTNHSNSSKEVPSSAVLRSLRVNVGPDGEETRAQTVQKSPEFLSTSESSSLLQDLQPSDSTSFILLNLTRAGLGSSAEHLVFVQDEAEDSGNDFLSSESTDSSIPWFLRVQELAHDSLIAATRAQLAKNAKTSSNGENVHLGSGDGQSKDSGPLPQVEKKLKCTVEGCDRTFVWPAHFKYHLKTHRNDRSFICPAEGCGKSFYVLQRLKVHMRTHNGEKPFMCHESGCGKQFTTAGNLKNHRRIHTGEKPFLCEAQGCGRSFAEYSSLRKHLVVHSGEKPHQCQVCGKTFSQSGSRNVHMRKHHLQLGAAGSQEQEQTEVLAEGSPRSLSSVPDVTHHLVTMQSGRQSYEVSVLTAVNPQELLNQGDLTERRT

SEQ ID NO: 60 - DNA, 12 bp, Artificial Sequence
  ZNF410 binding motif
catcccataatt

SEQ ID NO: 61 - DNA, 12 bp, Artificial Sequence
  ZNF410 binding motif
tatcccataata

SEQ ID NO: 62 - DNA, 12 bp, Artificial Sequence
  ZNF410 binding motif
cagcccataata

SEQ ID NO: 63 - DNA, 12 bp, Artificial Sequence
  ZNF410 binding motif
cataccataata

SEQ ID NO: 64 - DNA, 12 bp, Artificial Sequence
  ZNF410 binding motif
catcccgtaata

SEQ ID NO: 65 - DNA, 12 bp, Artificial Sequence
  ZNF410 binding motif
catcccataatg

SEQ ID NO: 66 - DNA, 12 bp, Artificial Sequence
  ZNF410 binding motif
caacccataata

SEQ ID NO: 67 - DNA, 12 bp, Artificial Sequence
  ZNF410 binding motif
cagaccataata

SEQ ID NO: 68 - protein, 31 aa, Artificial Sequence
  ZF1
KKLKCTVEGCDRTFVWPAHFKYHLKTHRNDR

SEQ ID NO: 69 - protein, 30 aa, Artificial Sequence
  ZF2
SFICPAEGCGKSFYVLQRLKVHMRTHNGEK

SEQ ID NO: 70 - protein, 30 aa, Artificial Sequence
  ZF3
PFMCHESGCGKQFTTAGNLKNHRRIHTGEK

SEQ ID NO: 71 - protein, 30 aa, Artificial Sequence
  ZF4
PFLCEAQGCGRSFAEYSSLRKHLVVHSGEK

SEQ ID NO: 72 - protein, 29 aa, Artificial Sequence
  ZF5
PHQCQVCGKTFSQSGSRNVHMRKHHLQLG

SEQ ID NO: 73 - protein, 478 aa, Mus musculus
MLSDELESKPELLVQFVQNTSIPLGQGLVESEAKDITCLSLLPVTEASECSRLMLPDETPNHANSSKEVPSSAVLRSLQVNVGPDGEETRAQTVQKSPEFLTTPESPSLLQDLQPSDSTSFILLNLTRAGLGSSAEHFVFVQDETEDSGADFLSAESTDSSIPWFLRVQELAHDSLIAATRAQLAKNAKTGSNGENVHLGSGDGQPKDSGPLPQAEKKLKCTVEGCDRTFVWPAHFKYHLKTHRNERSFICPAEGCGKSFYVLQRLKVHMRTHNGEKPFMCHESGCGKQFTTAGNLKNHRRIHTGEKPFLCEAQGCGRSFAEYSSLRKHLVVHSGEKPHQCQVCGKTFSQSGSRNVHMRKHHLQLGTTGSQEQDQTAEPLMGSSLLEDASVPNKNLVSMNSQSSLGGESLNLSNTNSILGVDDEVLTERASRPLSSVPDVTHHLVTMQSGRQSYEVSVLTAVNPQELLNQGDLTERQT

SEQ ID NO: 74 - protein, 9 aa, Artificial Sequence
  ZF1 fragment
VWPAHFKYH

SEQ ID NO: 75 - protein, 9 aa, Artificial Sequence
  ZF2 fragment
YVLQRLKVH

SEQ ID NO: 76 - protein, 9 aa, Artificial Sequence
  ZF3 fragment
TTAGNLKNH

SEQ ID NO: 77 - protein, 9 aa, Artificial Sequence
  ZF4 fragment
AEYSSLRKH

SEQ ID NO: 78 - protein, 9 aa, Artificial Sequence
  ZF5 fragment
SQSGSRNVH